(19) United States
(12) Patent Application Publication
(10) Pub. No.: US 2004/0247649 A1
(43) Pub. Date: Dec. 9, 2004

(54) Medicine-containing orally soluble films

(75) Inventors: Tony M. Pearce, Alpine, UT (US); Terry V. Pearce, Alpine, UT (US)

Correspondence Address: Parsons Behle & Latimer, Suite 1800, 201 South Main Street, P.O. Box 45898, Salt Lake City, UT, 84111, US

(73) Assignee: EdiZONE, LC

(21) Appl. No.: 10/845,597

(22) Filed: May 12, 2004

Related U.S. Application Data
(63) Continuation-in-part of application No. 10/325,721, filed on Dec. 20, 2002.
(60) Provisional application No. 60/469,703, filed on May 12, 2003.
(60) Provisional application No. 60/368,821, filed on Apr. 1, 2002.
(60) Provisional application No. 60/356,279, filed on Feb. 11, 2002.

(51) Int. Cl. A61K 9/68

(52) U.S. Cl. 424/440

(57) ABSTRACT

Various edibles, their compositions, and manufacturing methods therefore are disclosed. Some examples of the edibles include orally soluble films. Some of the films may have a pleasant taste, carry nutraceuticals, carry medication, or serve other purposes.

### CROSS REFERENCE TO RELATED APPLICATIONS

This patent application claims benefit of and priority to U.S. Provisional Patent Application Serial No. 60/469,703 filed on May 12, 2003. This patent application is a continuation-in-part of U.S. patent application Ser. No. 10/325,721 entitled “Snacks of Orally Soluble Edible Films” filed 20 Dec. 2002 (which is hereby incorporated by reference in its entirety), which claims benefit and priority to U.S. Provisional Patent Application Serial No. 60/368,821 filed on Apr. 1, 2002, and U.S. Provisional Patent Application Serial No. 60/356,279 filed on Feb. 11, 2002, and benefit of and priority to each of the foregoing is claimed.

### DETAILED DESCRIPTION

The detailed description is broken down by subheadings for the reader's convenience.

### Flavor Films and Film Base Generally

Various examples of snacks of orally soluble edible films, including their compositions and manufacturing techniques, are disclosed. The snacks may include one or more layers of film that is orally soluble and disintegrates quickly upon placement in a human mouth without leaving substantial residue that can be felt by the human tongue or which needs to be swallowed or ejected from the mouth

Snacks for human consumption providing flavor satisfaction and/or craving satisfaction, including snacks that utilize orally soluble edible films with high levels of appealing flavors and/or sweeteners and other ingredients are described in general and by way of specific examples below. The snacks use film that is orally soluble such that it disintegrates quickly upon placement in a human mouth without leaving substantial residue that can be felt by the human tongue or which needs to be swallowed or ejected from the mouth. The film may include at least one flavoring, the flavoring providing a strong flavor sensation to a person who places the snack on his or her tongue. A sweetener providing a sweetening sensation may also be used. The film may be one or more layers. Additional layers or materials may be provided to preserve the structure of the snack prior to use and/or to enhance or increase flavor sensations, and/or to provide a sensation of tartness, a sharp or sour flavor sensation. Such snacks avoid bulk, mess, noise when consumed, swallowing, and other disadvantages previously experienced by those persons interested in consuming snacks.

Snack Ingredients

Film-forming Ingredients

Orally soluble edible films can include many ingredients. The base ingredients for forming a film may include water (which can be later dried out of the film) and a film forming agent. Many other additives can be used as well for purposes of saliva stimulation, plasticizing, stabilizing, emulsifying, fillers, thickening, binding, coloring, or acting as a surfactant.

The film may include pullulan as a film-forming agent, and may also include water, additional film-forming agents, plasticizing agents, flavoring agents, acids, sulfur precipitating agents, saliva stimulating agents, cooling agents, surfactants, stabilizing agents, emulsifying agents, thickening agents, binding agents, coloring agents, sweeteners, fragrances, and the like. Further ingredients may be added to accomplish the purposes of the film, which in the prior art are limited to breath freshening, oral hygiene, and dispensing of medicaments and nutraceuticals. Highly-concentrated snack, candy, fruit or food flavors in an orally soluble edible film may be used to meet the needs of flavor satisfaction or craving satisfaction.

Film-forming agents can include pullulan, hydroxypropylmethyl cellulose, hydroxyethyl cellulose, hydroxypropyl cellulose, polyvinyl pyrrolidone, carboxymethyl cellulose, polyvinyl alcohol, sodium alginate, polyethylene glycol, xanthan gum, tragacanth gum, guar gum, acacia gum, arabic gum, polyacrylic acid, methylmethacrylate copolymer, carboxyvinyl polymer, amylose, high amylose starch, hydroxypropylated high amylose starch, dextrin, pectin, chitin, chitosan, levan, elsinan, collagen, gelatin, zein, gluten, soy protein isolate, whey protein isolate, casein, polysaccharides, natural gums, polypeptides, polyacrylates, starch, karaya gum, gelatin, mixtures thereof and others. A useful film former is pullulan, in amounts ranging from about 0.01 to about 99 wt %, about 30 to about 80 wt %, from about 45 to about 70 wt % of the film or from about 60 to about 65 wt % of the film.

Film forming agents which can be used within a pullulan-free edible film include, but are not limited to, cellulose ethers; modified starches; natural gums; gelatins; edible polymers; hydrocolloid flours; seaweed extracts; land plant extracts; derivatives thereof; and combinations thereof.

Examples of cellulose ethers include, but are not limited to, methylcellulose; ethylcellulose; hydroxymethylcellulose; hydroxyethylcellulose; hydroxypropylmethylcellulose; carboxymethylcellulose; derivatives thereof and combinations thereof.

Modified starch examples include, but are not limited to, acid and enzyme hydrolyzed corn and potato starches. Further, examples of natural gums include, but are not limited to, gum arabic; guar gum; locust bean gum; carageenan gum; acacia; karaya; ghatti; tragacanth agar; tamarind gum; xanthan gum; derivatives thereof; and combinations thereof.

Examples of edible polymers include, but are not limited to, microcrystalline cellulose; cellulose ethers; xanthan; derivatives thereof; and combinations thereof. Moreover, examples of hydrocolloid flour include, but are not limited to, guar gum; locust bean; microcrystalline cellulose; tara; derivatives thereof and combinations thereof.

Seaweed extract examples include, but are not limited to, alginates; carageenans; derivatives thereof; and combinations thereof. Land plant extract examples include, but are not limited to, konjac; pectin; arabinoglactan; derivatives thereof; and combinations thereof.

If the film has a relatively high oil content, it may be useful to avoid substantial amounts of humectant in the film (and more preferable to have no humectant in the film), so as to avoid producing an overly moist, self-adhering film. In particular, it may be useful to formulate the film with a plasticizing agent other than glycerin, which is also a humectant, and with a sweetener other than sorbitol, which is a mild humectant.

If desired, the effective amount of the film forming agent ranges from approximately about 10% to about 90%, more preferably 25% to about 75% dry weight of the film composition.

It should be appreciated by those skilled in the art, that other edible water-soluble film forming agents which exhibit desirable properties may be utilized. A bulk filler agent may be present, especially in pullulan free edible film compositions, to reduce the “slimy” texture of the compositions. The effective amount of the bulk filler agent can be as desired or range from approximately 10% to about 90%.

An example pullulan free edible film composition can include an effective amount of carageenan as a film forming agent; an effective amount of microcrystalline cellulose as a bulk filler agent; and an effective amount of polyethylene glycol as a plasticizing agent.

Additionally, to enhance the structure of the formed film, an effective amount of hydroxyethyl cellulose as a thickening agent can be incorporated

Binders

Useful binding agents include starch, in amounts ranging from about 0 to about 10 wt % or otherwise, and other binders known in the field.

Other examples include casein and pullulan.

Thickeners

To further enhance the structure of the film compositions, an effective amount of at least one thickening agent may be used. Suitable thickening agents include, but are not limited to, cellulose ethers, such as hydroxyethyl cellulose, hydroxypropylmethyl cellulose, or hydroxypropyl cellulose, either alone, or mixtures thereo. Other useful thickening agents include methylcellulose, carboxyl methylcellulose, and the like, in amounts ranging from about 0 to about 20 wt % or otherwise. Polymers are also useful thickeners, such as carbomer, polyvinyl pyrrolidone, carboxymethyl cellulose, polyvinyl alcohol, sodium alginate, polyethylene glycol, natural gums like xanthane gum, tragacantha, guar gum, acacia gum, arabic gum, water-dispersible polyacrylates like polyacrylic acid, methylmethacrylate copolymer, carboxyvinyl copolymers. The concentration of a water-soluble polymer in the final film can be as desired or can vary between 20 and 75% (w/w).

Fillers

A bulk filler agent may be added to the film forming agent for many purposes, including to reduce the “slimy” texture of the compositions. The effective amount of the bulk filler can vary, and can in some instances range from approximately 10% to about 90 by dry weight of the film composition. Suitable bulk filler agents include, but are not limited to, magnesium carbonate; calcium carbonate; calcium phosphate; calcium sulfate; magnesium silicate; aluminum silicate; ground lime stone; clay; talc; titanium dioxide; microcrystalline cellulose; cellulose polymers such as wood; derivatives thereof; and combinations thereof. The useful bulk filler agent of the present invention is microcrystalline cellulose.

Plasticizers

To improve flexibility and reduce brittleness of the edible film compositions, a softener, also known as a plasticizing agent, may be used. The effective amount of the plasticizing agent of the present invention can be as desired or range from approximately 0% up to about 20% dry weight of the film composition.

Suitable plasticizing agents of the present invention include, but are not limited to, polyols such as sorbitol; glycerin; polyethylene glycol; propylene glycol; monoacetin; diacetin; triacetin; hydrogenated starch hydrolysates; corn syrups; derivatives thereof; and combinations thereof. A polyalcohol may be used to achieve the desired level of softness of the film. Examples of polyalcohols include glycerol, polyethylene glycol, propylene glycol, glycerol monoesters with fatty acids or other pharmaceutically used polyalcohols. The concentration of the polyalcohol in the dry film may be as desired or can ranges between 0.1 and 5% (w/w). Some of the plasticizers are water soluble and are miscible with a polymer.

L-menthol can impart plasticization and this has been a beneficial side-effect for breath freshening films, but L-menthol can detract from the flavors of for example the fruit-flavored films of my inventions. Likewise, other plasticizing agents typically cited for film forming agents impart a bitter or otherwise very bad taste to the film, or, in the case of humectant plasticizers such as glycerin, attract moisture into the film in humid environments and cause the film pieces to undesirably adhere to adjacent film pieces. We have discovered that many of the highly-concentrated, oil-soluble flavors act as a plasticizer in their own right in the snacks, eliminating or substantially reducing the need for taste-detracting or bitter or bad tasting prior art plasticizers or humectant plasticizers.

We have discovered that many of the highly-concentrated, oil-soluble flavors that can be used in a snack act as a plasticizer in their own right in the snacks, eliminating or substantially reducing the need for taste-detracting or bitter or bad tasting prior art plasticizers or humectant plasticizers.

Some suitable plasticizers include, for example and particularly for oral-mucosal contact and other use in the oral cavity, glycerin, sorbitol, any of the glycols, polysorbate 80, triethyl titrate, acetyl triethyl titrate, and tributyl titrate.

Surfactants

Surfactants may optionally be included in the films. Surfactants can greatly reduce the surface tension of water when used in very low concentration. Note: Change the fonts in the following sentence to match the rest of the paragraph: They can also encapsulate or coat a material in a liquid. Useful surfactants include mono and diglycerides of fatty acids and polyoxyethylene sorbitol esters, such as, Atmos 300 and Polysorbate 80. When a combination of surfactants is used, the first component may be a polyoxyethylene sorbitan fatty acid ester or a .alpha.-hydro-.omega.-hydroxypoly (oxyethylene)poly(oxypropylene)poly(oxy-ethylene) block copolymer, while the second component may be a polyoxyethylene alkyl ether or a polyoxyethylene castor oil derivative. Other surfactants include Atmos 300, Polysorbate 80, Sorbitan fatty acid ester, pluronic acid, sodium lauryl sulfate, and the like. A surfactant can be added in a desired amount such as amounts ranging from about 0.5 to about 15 wt. In order to achieve esired instant wettability, the ratio between a first and second component of the binary surfactant mixture should be kept within 1:10 and 1:1, or within 1:5 and 1:3. The total concentration of surfactants in the final film depends on the properties of the other ingredients, but may stay between 0.1 and 5% (w/w).

Stabilizing Agents

Useful stabilizing agents include xanthan gum, locust bean gum and carrageenan, in amounts ranging from about 0 to about 10 wt % or otherwise. Other suitable stabilizing agents include guar gum and the like.

Cooling Agnt

Useful cooling agents include monomenthyl succinate, in amounts ranging from about 0.001 to about 2.0 wt % or otherwise. A monomenthyl succinate containing cooling agent is available from Mane, Inc. Other suitable cooling agents include WS3, WS23, Ultracool II and the like.

Emulsifiers

If desired, an emulsifier may be included in the film. An emulsifier may desired if the film includes oil which normally would not mix with the water component used in making the film. Emulsifiers may improve manufacturability and consistency of the film. Example emulsifying agents include casein, triethanolamine stearate, quaternary ammonium compounds, acacia, gelatin, lecithin, bentonite, veegum, and the like. Example amounts ranging from about 0 to about 5 wt % or otherwise.

Flavorings

The snacks can provide for craving-satisfying by including small amount of fats, oils, salt, meat flavorings or other flavorings without the customer ingesting significant amounts of those substances. Other unusual flavors may be provided as well, such as ‘apple pie ala mode’ or ‘roast beef with green peppers and onions’. By providing such flavors, the snacks can assist dieters in satisfying a craving without breaking a diet. Likewise, highly sour flavors desired by children can be provided without interfering with their appetite while not substantially lowering the pH of the film. Lowering the pH of the film may interfere with the film properties.

One aspect of the snacks is the use of more than one flavor in a film. Such a multiplicity of flavor within the mouth enhances the ingestion experience, in a similar way for example as caramel topping on vanilla ice cream. Not by way of limitation, this can be accomplished two ways: First, by using multiple feed streams of differently flavored films when casting or extruding the films; and second, by have a multiple-layer film with each layer of a different flavor. In the latter case, the film can be co-cast or co-extruded, or can be bonded together with a water soluble edible binder. For example, one layer can be caramel flavored and the other layer can be blackberry flavored. As another example, a single layer film can be striped with alternating layers that are raspberry flavored with a red color and cream flavored with a white color.

Cravings are often satisfied by fats or salts or substitutes or artificial flavoring along the same lines. An aspect of the snacks is the inclusion in an orally soluble edible film of any one or more of fats, salt, or substitutes or artificial flavoring providing a similar effect for flavor. For example, butter or cream can be included in the film. Or, an artificial butter flavor or artificial cream flavor. Examples of the use of cream flavors have been given above. Often these fats will be used in conjunction with other flavors as an enhancement. As another example, peanut oil or corn oil and salt can be included in a popcorn-flavored film for a more robust taste and better satisfaction of cravings. For the health conscious consumer, artificial butter flavoring and salt substitute such as potassium chloride can be used in the popcorn flavored film.

An aspect of the snacks is to provide for the satisfaction of cravings of, for example, dieters and health conscious consumers through orally soluble edible films containing meal and desert type flavorings and flavoring combinations. For example, apple pie ala mode has been described above. As another example, a concentrated roast beef flavor can be used in conjunction with a concentrated green pepper flavor and a concentrated onion flavor, either natural or artificial, for satisfaction of a craving for a main course of a meal. Another example is French fried potato flavored orally soluble edible film, with salt or salt substitute. The films are typically so thin that even if they contain real fat and salt, the portions are so small as to not create problems for the dieter or the health conscious consumer.

While water-based, spray-dried, alcohol/glycol-based, or semi-concentrated flavors can fulfill these aspects of the snacks, we have discovered that highly-concentrated oil-soluble flavors, whether natural or artificial or combinations thereof, can be satisfactory. Such highly-concentrated oil-soluble flavors provide sufficient flavoring to be considered intense, particularly in combination with the sweetening regimen provided below, even when delivered in the small film size suited for the mouth. The highly-concentrated, oil-soluble snack, candy, fruit or food flavors can provide the required intensity of flavor without the bitterness associated with attempts to obtain that level of flavor with water-based, spray-dried, alcohol/glycol-based, or semi-concentrated flavors. Further, since the oil-soluble flavor is not water soluble, it lingers in the mouth much longer, providing an extended period of flavor satisfaction or craving satisfaction. Typically these orally soluble edible films are brittle unless plasticized.

The flavorings that can be used include those known to the skilled artisan, such as natural and artificial flavors. These flavorings may be chosen from synthetic flavor oils and flavoring aromatics, and/or oils, oleo resins and extracts derived from plants, leaves, flowers, fruits and so forth, and combinations thereof. Representative flavor oils include: spearmint oil, cinnamon oil, peppermint oil, clove oil, bay oil, thyme oil, cedar leaf oil, oil of nutmeg, oil of sage, and oil of bitter almonds. Also useful are artificial, natural or synthetic fruit flavors such as vanilla, chocolate, coffee, cocoa and citrus oil, including lemon, orange, grape, lime and grapefruit and fruit essences including apple, pear, peach, strawberry, raspberry, cherry, plum, pineapple, apricot and so forth. These flavorings can be used individually or in admixture. Commonly used flavors include mints such as peppermint, wintergreen, spearmint, birch, anise and such fruit flavors, as cherry, lemon-lime, orange, grape, artificial vanilla, cinnamon derivatives, and others, whether employed individually or in admixture. Flavorings such as aldehydes and esters including cinnamyl acetate, cinnamaldehyde, citral, diethylacetal, dihydrocarvyl acetate, eugenyl formate, p-methylanisole, and so forth may also be used. Generally, any flavoring or food additive, such as those described in Chemicals Used in Food Processing, publication 1274 by the National Academy of Sciences, pages 63-258, may be used. Further examples of aldehyde flavorings include, but are not limited to acetaldehyde (apple); benzaldehyde (cherry, almond); cinnamic aldehyde (cinnamon); citral, i.e., alpha citral (lemon, lime); neral, i.e. beta citral (lemon, lime); decanal (orange, lemon); ethyl vanillin (vanilla, cream); heliotropine, i.e., piperonal (vanilla, cream); vanillin (vanilla, cream); alpha-amyl cinnamaldehyde (spicy fruity flavors); butyraldehyde (butter, cheese); valeraldehyde (butter, cheese); citronellal (modifies, many types); decanal (citrus fruits); aldehyde C-8 (citrus fruits); aldehyde C-9 (citrus fruits); aldehyde C-12 (citrus fruits); 2-ethyl butyraldehyde (berry fruits); hexenal, i.e. trans-2 (berry fruits); tolyl aldehyde (cherry, almond); veratraldehyde (vanilla); 2,6-dimethyl-5-heptenal, i.e. melonal (melon); 2-6-dimethyloctanal (green fruit); and 2-dodecenal (citrus, mandarin); cherry; grape; mixtures thereof; and the like.

The amount of flavoring employed is normally a matter of preference subject to such factors as flavor type, individual flavor, and strength desired. Thus, the amount may be varied in order to obtain the result desired in the final product. Such variations are within the capabilities of those skilled in the art without the need for undue experimentation. In general, amounts of about 0.1 to about 30 wt % are useable with amounts of about 2 to about 25 wt % being useful and amounts from about 8 to about 10 wt % are more useful.

Sweeteners

Artificial sweeteners are known to impart an aftertaste when used alone to impart sufficient sweetness to a sweetened film, and different sweeteners impart their peak sweetness at different times over the entire time that optimized flavors stay in the mouth. The snacks can provide a full level of sweetness desired without substantial aftertaste and providing at least some sweetness during the entire flavor experience.

Artificial sweeteners such as aspartame, acesulfame potassium, saccharine and sucralose can impart an aftertaste when used alone to impart sufficient sweetness to a sweetened film. In addition to this problem, different sweeteners impart their peak sweetness at different times after hydration with saliva. Use of one sweetener alone thus will not impart the sweetness desired over the entire time that optimized flavors stay in the mouth, being too early or too late. Prior films try to address this with a combination of two of these sweeteners. We have found that such combinations of two sweeteners must still be at such levels that an aftertaste is experienced, and do not give the desired sweetness over the full flavor experience. Thus a preferred aspect of the snacks is to use all three of these sweeteners in small amounts to achieve the full level of sweetness desired without substantial aftertaste and providing at least some sweetness during the entire flavor experience.

We have discovered that a tripartite admixture of three different sweeteners is highly effective at producing a desirable snack. Rather than mixing the sweeteners in equal amounts by weight or volume, we have found it particularly useful to mix them in equal amounts of sweetness potency.

Suitable sweeteners that can be included are those well known in the art, including both natural and artificial sweeteners. Suitable sweeteners include, e.g.:

A. water-soluble sweetening agents such as monosaccharides, disaccharides and polysaccharides such as xylose, ribose, glucose (dextrose), mannose, galactose, fructose (levulose), sucrose (sugar), maltose, invert sugar (a mixture of fructose and glucose derived from sucrose), partially hydrolyzed starch, corn syrup solids, dihydrochalcones, monellin, steviosides, and glycyrrhizin;

B. water-soluble artificial sweeteners such as the soluble saccharin salts, i.e., sodium or calcium saccharin salts, cyclamate salts, the sodium, ammonium or calcium salt of 3,4-dihydro-6-methyl-1,2,3-oxathiazine-4-one-2,2-dioxide, the potassium salt of 3,4-dihydro-6-methyl-1,2,3-oxathiazine-4-one-2,2-dioxide (acesulfame-K), the free acid form of saccharin, and the like;

C. dipeptide based sweeteners, such as L-aspartic acid derived sweeteners, such as L-aspartyl-L-phenylalanine methyl ester (aspartame) and materials described in U.S. Pat. No. 3,492,131, L-alpha-aspartyl-N-(2,2,4,4-tetramethyl-3-thietanyl)-D-alaninamide hydrate, methyl esters of L-aspartyl-L-phenylglycerin and L-aspartyl-L-2,5,dihydrophenyl-glycine, L-aspartyl-2,5-dihydro-L-phenylal-anine, L-aspartyl-L-(1-cyclohexyen)-alanine, and the like;

D. water-soluble sweeteners derived from naturally occurring water-soluble sweeteners, such as a chlorinated derivative of ordinary sugar (sucrose), known, for example, under the product description of sucralose; and

E. protein based sweeteners such as thaumatoccous danielli (Thaumatin I and II).

Other sweeteners may be used as well.

Sour Snacks

One difficulty in making good tasting flavor films is in providing highly sour flavors, for example sour apple or sour lemon or sour peach. Sourness, or tartness, is generally caused by low pH. Low pH is known to adversely effect nearly all of the film forming ingredients, causing for example brittleness, low tensile strength, and short shelf life. One solution that we have discovered is to combine the snack, candy, fruit or food flavors, the acid required to cause the desired tartness, and pullulan (as a film forming ingredient), since this combination is less effected (though still somewhat effected) than other combinations which use as the film former starches or gums. Another solution we have discovered is to have the acid in a separate layer from the film. For example, as the film is drying, the acid can be applied in powder form (optionally diluted by a filler material such as maltodextrin) to the exterior of the film (optionally with a binder material to enhance the adhesion to the film). In this way, the acid does not penetrate the film itself to a degree great enough to deteriorate the film. This discovery is particularly exciting because the acid in this case may be the first thing to contact the tongue, so the burst of sour is first even before the flavor, which is desirable in many types of candy snacks. We have also discovered a way to make the sour flavor last longer. This is done through the same method of having the acid in a separate layer, except that some or all of the acid is microencapsulated to create a delay in salvation. Using a mixture of acids with different degrees of delay in solvation can make the sour flavor last. The sour flavor can last at least as long as the highly-concentrated oils which linger in the mouth. The acid can also be put in other locations than on the exterior. For example, it can be put between two layers of film which are bound together by the binding agent that is with the acid. In this case, the film flavor would be tasted first, then the sour, which is desirable in some instances. Common edible acids are citric, tartaric and malic, and we have found that tartaric acid is especially advantageous, especially when it is an ingredient of the film rather than coated on the exterior. Tartaric acid provides considerable acidity for the amount of volume occupied, which in such thin films is advantageous.

Effervescence

The snacks can provide entertainment or whole-mouth disbursal to the film by means of effervescence. Effervescence, when the snack is dissolved in the mouth, can be both in a quiet bubbly way and in a loud snappy way. Multiple types of film (layers or sections) or multiple flavors of film, for example raspberries and cream, within a single oral dose can be provided.

An optional aspect of the snacks is the inclusion of the property of effervescence. Advantages of effervescence include without limitation the delight of children and the more effective coating of the oral cavity with a medicament. The effervescence can be in the form of loud snappy bubbles or quiet foamy bubbles or any combination thereof.

Loud snappy bubbles are obtained by the inclusion of small bits of a gas-releasing agent in the snack.

Gasified candy is usually hard candy containing gas, such as carbon dioxide. Such a candy may be made by a process which comprises melting crystalline sugar, contacting such sugar with gas at a pressure of 50 to 1,000 psig for a time sufficient to permit incorporation in said sugar of 0.5 to 15 cc of gas per gram of sugar, maintaining the temperature of said sugar during said absorption above the solidification temperature of the melted sugar, and cooling said sugar under pressure to produce a solid amorphous sugar containing the gas. Upon the release of the pressure, the solid gasified candy fractures into granules of assorted sizes.

The resultant product may contain 1% to 4% water and most typically 2% to 3% water by weight of the total composition. (All figures expressed herein as a percentage are in terms of weight percent, unless specifically expressed to the contrary.) Lower levels of moisture may not be practicably obtainable because the additional heat necessary to drive off the water causes the candy melt to caramelize or burn, resulting in an off-flavor, undesirable product. High moisture levels result in a soft, sticky matrix which rapidly liberates the entrapped gas and is thus not storage stable.

The gasified candy, when placed in the mouth, produces an entertaining but short-lived popping sensation. As the candy is wetted in the mouth the candy melts and the gas escapes. The tingling effect in the mouth is sensational but short.

When the solidified gasified candy is fractured by the release of pressure from the preparation vessel, the resultant granulated pieces are irregular, randomed-sized pieces having the appearance of pieces of broken glass or what might be termed sharp-faced pieces of gravel. The granulated pieces are sieved to provide the gasified candy in a range of particle sizes.

A process may be used wherein the sugar is maintained above its fusion temperature of 25 degrees to 200 degrees. C. (77 degrees to 392 degrees F.) while the gas, at a pressure of 50-1000 psig, is contacted with the sugar melt for 2 to 6 minutes. Mixtures of sugar are held at 150 degrees or 160 degrees. C. (302 degrees or 320 degrees F.) while carbon dioxide at 600 psig is mixed into the liquid sugar for a short period of time, usually about six minutes. Example processes utilize a sugar melt maintained at 315 degrees to 325 degrees F. while carbon dioxide at 600 psig is brought into contact with the sugar.

In producing gasified candy by a commercial process conducted in accordance with this disclosures, gasified candy may be produced from a mixture of sucrose, lactose and corn starch dissolved in water and evaporated to a sugar melt containing about 2 to 3% water. Carbon dioxide may be maintained at 625 to 675 psig in the pressure vessel containing the sugar melt while the vessel contents are mixed for 4 to 6 minutes. The sugar melt may be held at about 295 degrees. F. during the gasification. Following the gasification, the gasified melt may be transferred to a water-jacketed cooling tube where it is held for 21/2-3 hours at a pressure which is 50 psi higher than the gasification pressure. At the end of the cooling cycle, the pressure may be released from the cooling tube which causes the solidified gasified candy to shatter into multiple fragments.

As used herein “observable gas bubbles” means those gas bubbles which can be seen when the solidified gasified candy is observed through light microscopy. The observable bubbles are generally classified into large bubbles, i.e., those having a diameter of about 100 mu. and above and small bubbles, i.e., those having a diameter below about 100 mu. The small bubbles are generally not considered to be significant since they do not contribute to the popping sensation.

Gasified candy which produces a more pronounced popping sensation can be prepared by utilizing low preparation temperatures. The gasified candy product obtained by this improved process contains observable gas bubbles having a maximum diameter substantially larger than those in the commercial product produced heretofore. For example, observable large gas bubbles with a diameter in the range of about 150 to about 225 mu. may be produced, or large gas bubbles with a diameter above about 225 mu. and more particularly in the range of about 300 to about 350 mu.

A sugar melt may be gasified at superatmospheric pressure and the gasified sugar melt is cooled below its fusion temperature under superatmospheric pressure to form a gasified candy. In particular, steps utilized may include maintaining the sugar melt during the gasification at a temperature effective to produce in the gasified candy product observable gas bubbles wherein the majority of the large observable gas bubbles have a diameter of above about 225 mu.

The product may include a gasified candy comprising a solidified fused sugar containing therewithin bubbles of gas, said gas being at superatmospheric pressure and said observable bubbles having a maximum diameter of about 350 mu.

The gasified candy component of the snack may be a hard sugar product having bubbles of gas entrapped therein and is conveniently produced as described below or otherwise. The gasified candy can be prepared from any of the commercially-available sugars employed in the confectionary industry. Thus, such sugars as glucose, fructose, sucrose, lactose and the like, alone or in combination, may be employed. A mixture of sucrose with corn syrup (containing glucose, maltose, dextrin) may also be satisfactory. A mixture of sucrose, lactose and corn syrup in a weight ratio of 52:27:21 may be useful, providing a gasified hard product particularly characterized by its good gas retention and reduced stickiness on standing. A 40:40:20 mixture of sucrose, lactose and corn syrup may also be useful since it additionally should provide good high temperature stability. The moisture content of the gasified candy should be between 1.0 and 5.0% or between 2.0 and 3.0%.

The gases used to prepare the gasified candy may be any of the commonly-available gases which are substantially unreactive with the sugar or sugars being employed and include such gases as carbon dioxide, nitrogen or air, but carbon dioxide is useful.

The gasified candy may be employed in granulated form and is most easily provided in that form following the solidification of the gasified sugar in the preparation vessel or in a separate cooling tube provided for this purpose. The release of pressure from the vessel fractures the gasified sugar into granulated pieces in a wide range of assorted sizes. Although finely divided pieces of gasified candy in a variety of sizes may be employed, the pieces may be sieved to provide uniform sized pieces. In general, smaller sized pieces may be more useful. Gasified candy having a particle size distribution of −4 to +40 U.S. Sieve Series can be employed, or from −4 to +14 U.S. Sieve Series.

A sugarless form of gasified candy can also be employed by substituting sugarless material, such as sorbitol, for the sugar in the gasified candy preparation described herein.

The gasified candy may be prepared in accordance with the examples and steps described below or otherwise.

The sugar or mixtures of sugars are placed in a heated vessel provided with a mixer. A small quantity of water is added to dissolve the sugar and other additives. Heat is applied to the vessel sufficient to dissolve the sugars. The mixture is then evaporated to produce a melt having 1-5%, preferably 2-3%, of water. The required amounts of coloring, flavoring and/or active ingredient may be added when the desired water content is achieved. The evaporation may be conducted at atmospheric pressure or, preferably, under a vacuum of up to 15 inches of mercury. Melt temperatures of between about 280 degrees to 320 degrees F. may be necessary to reach the desired moisture content. Care should be exercised to prevent caramelizing the sugary mix.

The next step is to gasify the sugar melt and it is in this particular step that the temperature of the sugar melt is critical when practicing the process described herein. The temperature at which the melt is maintained while the gas is being incorporated therein affects the “pop” of the product, i.e., the quality of the popping sensation produced by the gasified candy. Lower melt temperatures promote larger gas bubbles because both the viscosity and the surface tension of the melt is increased as the temperature is reduced and bubble diameter is proportional to both viscosity and surface tension. Melt temperatures that produce observable gas bubbles having a maximum diameter of about 350 mu. are satisfactory. Maximum melt temperatures of about 280 degrees F. should achieve this desired result and produce a product with a good “pop”. The larger the gas bubbles entrapped in the solidified candy, the more pronounced the popping when the candy is eaten. Observable bubbles having a maximum diameter of about 350 mu may be produced when the melt temperature during gasification is below about 280 degrees F. and the mixing time is about 4-6 minutes.

The sugar melt is gasified by introducing gas, such as carbon dioxide, at superatmospheric temperature into the closed vessel. Pressures of about 50-1000 psig, preferably about 500-700 psig, are utilized. While the required amount of gas is being introduced into the closed vessel, the liquid melt is agitated to effect intimate contact between the gas and the melt. Sufficient gas is incorporated into the melt to provide 0.25-7.0 cc. of gas per gram of candy in the final product. The gasification is completed within a fairly short period of time, such as than about 10 minutes or 2 to 6 minutes. During this time, the melt is maintained at a maximum temperature as explained above. The required amount of mixing may be readily determined by those skilled in the art. For example, using a Parr bomb (a small pressure vessel equipped with a mixer) to prepare about 1,000 grams of melt, mixing speeds of 200-1500 rpm, preferably 500-900 rpm, may be used.

The gasified melt is then permitted to solidify while maintaining the vessel under pressure. Where desired, the solidification may be conducted in a cooling tube. Bubbles of gas are entrapped in the solidified sugar. After the melt has solidified, the pressure is released, which fractures the sugary mass into granulated pieces of a variety of sizes.

The following example is illustrative.

Sucrose, lactose and corn syrup in a weight ratio of 52:27:21 were dissolved in water and evaporated at a temperature of 320 degrees F. atmospherically to produce a melt having a moisture content of about 3%. This melt was placed in a pre-heated Parr bomb (a small pressure vessel equipped with a mixer). The bomb was placed in a controlled temperature bath and pressurized with carbon dioxide at 750 psig for five minutes during which time the sugar melt was mixed vigorously. Following the carbonation, the pressure was maintained while the vessel was cooled so as to solidify the sugar melt. Rapidly releasing the pressure fractured the solidified carbonated candy into multiple pieces of various sizes.

For example, the bits of candy can be inserted into an encapsulant film made of orally soluble edible film in means as described above. As another example, the bits of pressurized-gas candy can be coated on the film, either as it dries or by use of a binder. As another example, the bits of pressurized-gas candy can be put between layers of orally soluble edible film. The flavor in the film can be the same or different than the flavor in the pressurized-gas candy. The advantage of combining pressurized-gas candy with film, as opposed to having the bits of candy sold in a pourable envelope, include lack of messiness and the ability to put a consistent pre-measured amount into the oral cavity each time. When the film so made is placed into the oral cavity, the pressurized-gas candy is solvated to release the pressurized gas, and loud snappy effervescence results. This can be very entertaining to the user. The second type of effervescence is the quiet foamy type. This can be obtained by mixing ingredients such as sodium bicarbonate and citric acid in the presence of water. Since sodium bicarbonate and citric acid are both dry, they can co-exist in the same film, and when water (saliva) is added, gas bubbles will form. One example is to make a two layer film, wherein the top layer contains sodium bicarbonate and the bottom layer contains citric acid. The two layers can be joined with a binder. Another example is to have a one-layer film containing sodium bicarbonate, and put a dry coating of citric acid on one or both sides of the film. This will provide the consumer with an often-desired sour taste, followed promptly by effervescence.

Film Structure and Shape

Film used in the snacks may be of a variety of structures and shapes as described herein or otherwise.

### EXAMPLE STRUCTURES

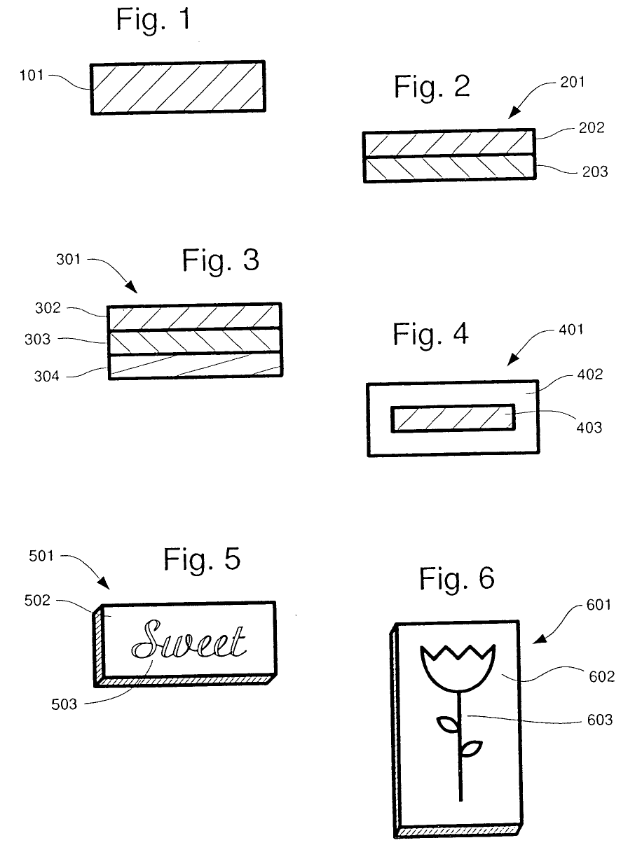

Referring to FIG. 1, a single layer film 101 of generally homogenous material is depicted. The film 101 may or may not include an exterior coating (not shown), such as an acid to effect a sour taste, a powder to reduce tackiness, or another coating.

Referring to FIG. 2, a film 201 having a first layer 202 and a second layer 202 of like or different films laminated, bonded or lain together is depicted.

Referring to FIG. 3, a three-layer film 301 is depicted having a first layer 302, a second layer 302 and a third layer 303. The layers may be the same or a different material.

Referring to FIG. 4, a cross-sectional view of a snack 401 is provided that includes a film 402 encapsulating another substance 402.

Referring to FIG. 5, a view of a snack 501 is provided that includes a film 502 acting as a substrate for lettering or printing 503. The lettering 503 may be atop the film 502 or embedded or embossed or cast or molded or stamped or located within it.

Referring to FIG. 6, a snack 601 is depicted that includes a film 602 having a graphic image or FIG. 603 on it.

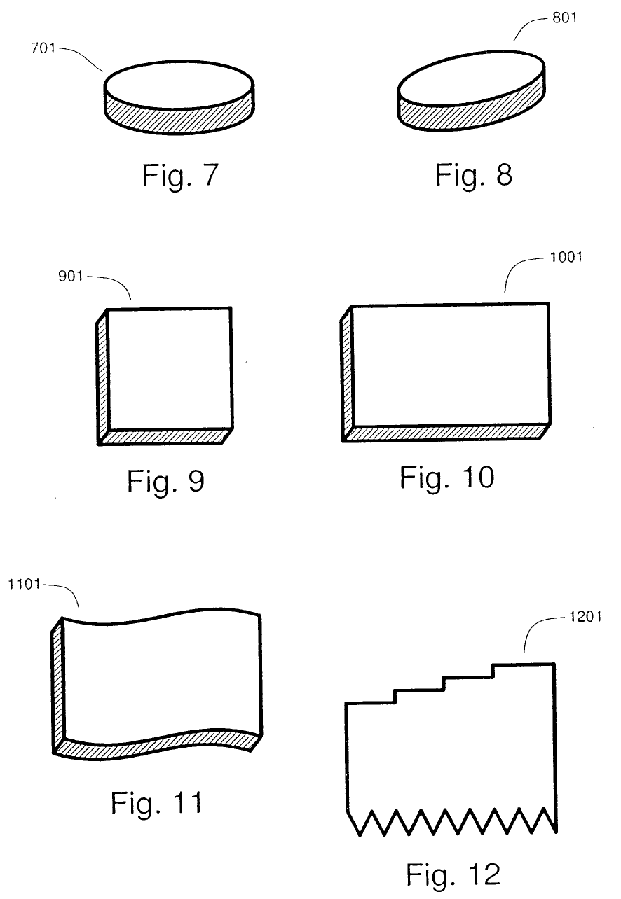

Referring to FIG. 7, a film 701 is depicted having a round disc shape.

Referring to FIG. 8, a film 801 is depicted having an oval or elliptical shape.

Referring to FIG. 9, a film 901 is depicted having a square shape.

Referring to FIG. 10, a film 1001 is depicted having a rectangular shape.

Referring to FIG. 11, a film 1101 is depicted having a curved or wavy shape.

Referring to FIG. 12, a film 1201 is depicted having both a stepped and a jagged edge.

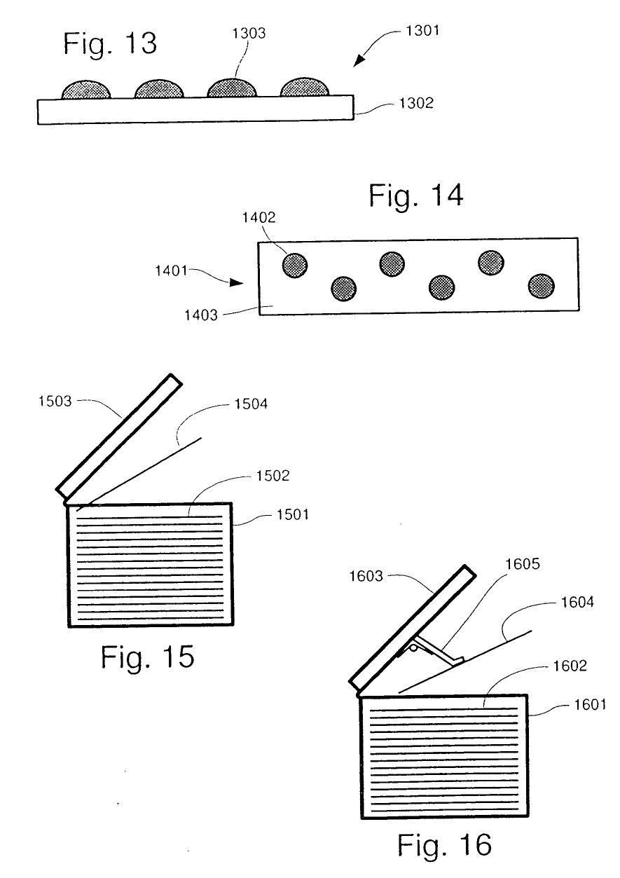

Referring to FIG. 13, a snack 1301 is depicted that includes a film 1302 and gas-releasing candy 1303 located thereon.

Referring to FIG. 14, a snack 1401 is depicted that includes a film 1402 and gas-releasing candy 1403 located therein.

### ENCAPSULATION

The snacks can also provide for encapsulation of other non-film materials. Encapsulating non-film edible materials such as flavored oils, medicaments, breath fresheners, antiseptic, antimicrobial, nutraceuticals, candy, and the like can be achieved through layering or through a containment chamber within the snack.

An aspect of the snacks is the use of orally soluble edible films for encapsulation of other materials which are desirably output into the oral cavity when the encapsulating film dissolves. This is different than the prior art method of encapsulating medicament pills or neutraceutical pills with edible soluble coatings, since in those cases the intent of the coating is to prevent the medicament or neutraceutical from being released in the oral cavity, but allow its release in the stomach or intestine. In the case of the snacks, it is the intention to allow the saliva to dissolve away the film encapsulant and release the contents into the oral cavity. Not by way of limitation, the contents could be flavored, sweetened vegetable oil for flavor satisfaction or craving satisfaction or special flavor effects such spicy hot or sour, or medicaments intended for the oral cavity, or breath fresheners, or candy of any type, or fats or creams. For example, the film encapsulant can be an orally soluble edible film of the snacks as described above with an apple pie and cinnamon flavor, and the contents can be a cream type candy. When placed in the oral cavity, the consumer first tastes the apple pie, and then when the film dissolves to release the contents, tastes the cream. In this way the consumer gets a sensation similar to apple pie ala mode. As another example, the orally dissolvable edible film encapsulant may have a pleasant sweetened spearmint taste, and the contents may contain essential oils such as menthol or peppermint. In this way the user experiences a pleasant candy-like mint flavor and ends with a fresh-feeling mouth and the sensation of fresh breath. Encapsulation can take many forms, and the following examples are not by way of limitation. The film can be formed into an envelope, the contents placed within, and the envelope flap closed by tucking or sealing, such as heat sealing or use of a binder for sealing. Or, the film can be formed into a pouch, the material placed into an open end, and the pouch sealed closed. Or, the film can be directly coated onto the contents using pill coating techniques. Or, the film can be made into a two piece capsule such as those used to contain neutraceuticals or medications, such as a specially formulated gelatin capsule, the contents inserted, and the two pieces put together. Or, the film can be made whole around a liquid center by using the known techniques and equipment used to make for example vitamin E capsules.

Avoiding Medical Taste

Some films in the past utilized L-Menthol as a plasticizer necessary for successful film formation. L-Menthol is also a breath freshener which if used in snacks would detract from and interfere with their pleasant flavor. The snacks described herein may be made generally L-Menthol free to avoid a medicinal taste.

Flavor Release Rate

Some of the snacks described herein will have a flavor release rate of that does not exceed the snack solubility rate, so that the snack will have flavor the entire time that it is being dissolved in the mouth. Other flavor release ates are possible.

Film or Snack Shape

If desired, the perimeter of the snack may be shaped other than traditional square or rectangular shapes. For example, shaping the film in the form of animals or stars for the delight of children. Or, for example, shaping the film into a company logo or movie character for promotional or advertising purposes. Or, for examples shaping the film into numbers and letters to be used by children in play much in the same way that the letters in alphabet soup are used. Or, for example, to differentiate one type of film-based medicament from another, just as prescription medications come in unique shapes to differentiate one medicament from another. Not by way of limitation, in flat films, this may be accomplished by standard die cutting techniques. In many cases, the left-over film from the die cutting process can be rehydrated and used again to make new film, thus making the process cost efficient.

An aspect of the snacks is to go beyond flat films into three dimensional films. This can have the same advantages as cited for films with non-rectangular perimeter shapes. This may be done with films in which the film forming agent or a binder thereof has thermoplastic properties. Pullulan is an excellent example of an orally soluble edible film former with thermoplastic properties. This can be accomplished by post-forming the flat film by methods including compression forming and embossing, or by other means used for forming thermoplastics including extrusion and injection molding. Delight, entertainment, education, promotion, and differentiation can be achieved by selection of shapes. For example, the films can be shaped into various types of dinosaurs and sold as candy, educating children as to the different types of dinosaurs.

Artwork

Images

In order to increase the attractiveness of the snacks to the consumer, they may include an exterior surface with an embossed, sculpted, sprayed or printed image, figure, logo, text, graphics, characters, art or words. Edible ink may be used for such optical images. Such optical representations may be related or unrelated to the flavor that the snack provides. Such optical representations may be chosen to cause the consumer to experience pleasant thought when consuming the snack, to attract the consumer's attention in a retail location, for advertising, education other related or unrelated products, or for other reasons. In general the optical images are provided to make the product more entertaining and delightful than it would be without the optical images. The snacks described herein provide an aesthetic and pleasing appearance through the use of bright and/or multiple colors. The films may be brightly colored and entertaining. Multiple colors may be used including those that intentionally color the human tongue. Glow in the dark materials may be included in order to make the tongue glow in darker locations, such as at dances, in bars, etc. Printing is desirable for other reasons as well. The promotional and advertising industries will benefit by placing logos and other promotional and advertising on the film. Sweepstakes and contest winners could find their prize on the film. Comic strips could be printed on the film, especially on continuous roll-type film. Depending on the type of film, particularly for medicament films or neutraceuticals films, it may be important to print instructions and warnings on the film. Printing, particularly in multiple colors, can also provide visual stimulation, delight, and/or entertainment, especially to youngsters. For example, characters from a loved television show or movie can be printed on the film. The film can be more educational and entertaining by inclusion of for example riddles and questions, with the answer on the next piece of film or further down the roll or on the other side of the film. It may be important to put trademarks on the film. It may be desirable to print the name of the product on the film to aid in increasing brand awareness. The film can be printed on one side or both sides.

Printing of text, graphics, photographs or combinations thereof onto orally soluble edible films, including but not limited to the snack, candy, fruit or food flavored films may be accomplished through standard printing techniques in combination with the use of edible inks. Alternatively, it may be accomplished with different colored films melded together. Not by way of limitation, two types of inks may be employed. The first type is an ink made from a solution containing the same film forming ingredient as in the orally soluble edible film being printed upon, to which dye or pigment is added. The second type are commonly available vegetable dies. The printing can be done in one or more colors, and is may done while the film is in roll form (prior to being cut for individual packaging) or as it comes off the film line and is heading toward the roller. The ink can be flavorless, or can add more of the same type of flavor as is in the film, or can add one or more additional flavors, or can add salivary stimulants which will help produce saliva to dissolve the film, or can add tartness or sourness. This last is important since tartness is created by the addition of acid, which if put into the film itself, can harm the desired physical properties of the film. This is true of most film bases. Printed on the exterior of the film, the acid will provide the initial sour flavor burst desired by many children and other consumers, but will not adversely affect the film.

Coloring

The snacks can use bright or multiple colors, as compared with the muted colors of the prior art. While these may be printed onto the film as previously described, the pigment or dye may be included within the film itself in sufficient intensity to be bright and pleasing to the eye. Further, multiple colors, whether bright or muted, can be used within the same film. Not by way of limitation, this can be accomplished two ways: First, by using multiple feed streams of different colors when casting or extruding the films; and second, by have a multiple-layer film with each layer of a different color. In the latter case, the film can be co-cast or co-extruded, or can be bonded together with a water soluble edible binder. While in most films it would not be desirable for the film to leave color in the mouth, a further aspect of the snacks especially for children's films is to use the type of dye, such as vegetable dyes, that leave the child's tongue and/or teeth colored after ingestion.

The compositions of the snacks can also contain coloring agents or colorants. The coloring agents are used in amounts effective to produce the desired color. Some coloring agents include pigments such as titanium dioxide, may be incorporated in amounts of up to about 5 wt % or more. Colorants can also include natural food colors and dyes suitable for food, drug and cosmetic applications. These colorants are known as FD&C dyes and lakes. The materials acceptable for the foregoing spectrum of use could be water-soluble, and include FD&C Blue No. 2, which is the disodium salt of 5,5-indigotindisulfonic acid. Similarly, the dye known as Green No. 3 comprises a triphenylmethane dye and is the monosodium salt of 4-[4-N-ethyl-p-sulfobenzylamino) diphenyl-methylene]-[1-N-ethyl-N-p-sulfonium benzyl)-2,5-cyclo-hexadienim-ine]. A full recitation of all FD&C and D&C dyes is available in industry literature.

Medicament

In various films it may be desirable to include ingredients other than mere sweeteners and flavoring, such as a bactericide, antiseptic, antimicrobial, stimulant, or other medicament. The fast dissolving film may include at least one physiologically acceptable, pharmaceutically active agent. The expression “physiologically acceptable” as used herein is intended to encompass compounds, which upon administration to a patient, are adequately tolerated without causing undue negative side effects. The expression encompasses edible compounds.

The expression “pharmaceutically active agents” as used herein is intended to encompass agents other than foods, which promote a structural and/or functional change in and/or on bodies to which they have been administered. These agents are not particularly limited; however, they should be physiologically acceptable and compatible with the film. Suitable pharmaceutically active agents include, but are not limited to:

A. antimicrobial agents, such as triclosan, cetyl pyridium chloride, domiphen bromide, quaternary ammonium salts, zinc compounds, sanguinarine, fluorides, alexidine, octonidine, EDTA, and the like,

B. non-steroidal anti-inflammatory drugs, such as aspirin, acetaminophen, ibuprofen, ketoprofen, diflunisal, fenoprofen calcium, naproxen, tolmetin sodium, indomethacin, and the like,

C. anti-tussives, such as benzonatate, caramiphen edisylate, menthol, dextromethorphan hydrobromide, chlophedianol hydrochloride, and the like,

D. decongestants, such as pseudoephedrine hydrochloride, phenylepherine, phenylpropanolamine, pseudoephedrine sulfate, and the like,

E. anti-histamines, such as brompheniramine maleate, chlorpheniramine maleate, carbinoxamine maleate, clemastine fumarate, dexchlorpheniramine maleate, diphenhydramine hydrochloride, diphenylpyraline hydrochloride, azatadine meleate, diphenhydramine citrate, doxylamine succinate, promethazine hydrochloride, pyrilamine maleate, tripelennamine citrate, triprolidine hydrochloride, acrivastine, loratadine, brompheniramine, dexbrompheniramine, and the like,

F. expectorants, such as guaifenesin, ipecac, potassium iodide, terpin hydrate, and the like,

G. anti-diarrheals, such a loperamide, and the like,

H. sub.2-antagonists, such as famotidine, ranitidine, and the like; and

I. proton pump inhibitors, such as omeprazole, lansoprazole, and the like,

J. general nonselective CNS depressants, such as aliphatic alcohols, barbiturates and the like,

K. general nonselective CNS stimulants such as caffeine, nicotine, strychnine, picrotoxin, pentylenetetrazol and the like,

L. drugs that selectively modify CNS function such as phenyhydantoin, phenobarbital, primidone, carbamazepine, ethosukimide, methsuximide, phensuximide, trimethadione, diazepam, benzodiazepines, phenacemide, pheneturide, acetazolamide, sulthiame, bromide, and the like,

M. antiparkinsonism drugs such as levodopa, amantadine and the like,

N. narcotic-analgesics such as morphine, heroin, hydromorphone, metopon, oxymorphone, levorphanol, codeine, hydrocodone, xycodone, nalorphine, naloxone, naltrexone and the like,

O. analgesic-antipyretics such as salycilates, phenylbutazone, indomethacin, phenacetin and the like,

P. psychopharmacological drugs such as chlorpromazine, methotrimeprazine, haloperidol, clozapine, reserpine, imipramine, tranylcypromine, phenelzine, lithium and the like.

The amount of medicament that can be used in the rapidly dissolving films is dependent upon the dose needed to provide an effective amount of the medicament.

The films that deliver a medicament can also include a triglyceride. Examples of triglycerides include vegetable oils such as corn oil, sunflower oil, peanut oil, olive oil, canola oil, soybean oil and mixtures thereof. The triglyceride may be added to the film in amounts from about 0.1 wt % to 16 about 12 wt % or otherwise.

The films that contain medicaments also can include a preservative. The preservative may be added in amounts from about 0.001 wt % to about 5 wt %, preferably from about 0.01 wt % to about 1 wt % of the film. Useful preservatives include sodium benzoate and potassium sorbate.

The medicament containing films can also include a polyethylene oxide compound. The molecular weight of the polyethylene oxide compound can range from about 50,000 to about 6,000,000. A useful polyethylene oxide compound is N-10 available from Union Carbide Corporation. The polyethylene oxide compound may be added in amounts from about 0.1 wt % to about 5 wt % or otherwise.

The medicament containing films can also include propylene glycol. The propylene glycol may be added in amounts from about 1 wt % to about 20 wt % or otherwise.

The active ingredient used in the film can be coated to mask the taste of the active ingredient or to prevent the active ingredient from numbing the tongue or other surfaces in the oral cavity. The coatings that can be used are known to those skilled in the art. These include polymers such, as Eudragit.RTM., cellulosics, such as ethylcellulose, and the like.

An additional way to mask the taste of the active ingredient is by using an ion exchange resin such as Amberlite RP-69, available from Rohm and Haas, and Dow XYS-40010.00, available from the Dow Chemcial Co.

Sulfur Precipating Agents

Sulfur precipitating agents that reduce oral malodor can also be added to the films. These agents bind with, and inactivate, the volatile sulfur compounds that cause a large percentage of oral malodor. Sulfur precipitating agents that may be useful include metal salts such as copper salts and zinc salts. Useful salts include copper gluconate, zinc citrate and zinc gluconate. The amount of sulfur precipitating agent may be from about 0.01 to about 2 wt %, from about 0.1 5 wt % to about 1.5 wt %, or from about 0.25 wt % to about 1.0 wt % of the film.

Saliva Stimulating Agents

Saliva stimulating agents can also be added to the films. Saliva stimulating agents include food acids such as citric, lactic, malic, succinic, ascorbic, adipic, fumaric and tartaric acids. Useful food acids are citric, malic and ascorbic acids. The amount of saliva stimulating agents in the film may be from about 0.01 to about 12 wt %, from about 1 wt % to about 10 wt %, or from about 2.5 wt % to about 6 wt %, or otherwise.

Film or Snack Packaging

Various containers are provided that provide easy dispensation of the snacks and can provide advertising, labeling, logos, cartoon characters or other information or serve other functions

Breath freshening and oral care films have been marketed in flat containers with a flip top that snaps shut. This is a non-bulky solution to putting a short stack of film pieces in a pocket or purse. However, this type of container does not meet the needs or wants of users in many situations. One problem is that the films are stacked, and sometimes become lightly adhered to one another, especially in humid environments. Even when not adhered, it can be difficult to slide the film out of the container while leaving the other films behind, because to be successful the method requires proper technique, dexterity, and involves the use of the finger which will vary in its friction characteristics from person to person and day to day, and will be seen as non-hygienic by some. New dispensers for orally soluble edible films can be provided which have one or more of the following functions as compared with prior flat flip-top containers: greater ease or reliability of dispensing, an aesthetically pleasing experience, a more entertaining experience, a more hygienic experience, or the ability to promote, advertise, educate, warn and/or instruct. Greater ease or reliability of dispensing can be provided in a number of ways. First, the dispenser can be designed to operate in a similar way as the prior dispensers which offer the films in discrete pieces in a stack, but have a mechanism with a mechanical ‘finger’, perhaps with a rubber-clad tip, which drags across the top film in the stack an removes it from the container. In this way, the friction is increased as compared to a finger, the technique is designed into the mechanism and does not require human skill and can be optimized for repeatability, and since the finger does not enter the container (but rather picks off the piece of film after most of it is out of the container), the process is more hygienic. This mechanism can automatically activate as the lid is opened, so that each time the lid is opened a piece of film is offered. The mechanism can be other than a mechanical finger. It may be thus desired to use of a mechanism to easily, repeatably and hygienically offer a piece of film from a stack of films to the consumer. Or the film can be packaged in roll form instead of in discrete pieces. The film can then be dispensed much as a roll of stamps in a standard stamp dispenser or as cellophane adhesive tape from a standard tape dispenser. The film can either be perforated for separation at specific intervals, or a cutting/breaking mechanism can be included in the container, or the film can be made intentionally brittle enough to be broken off, or the tape can be bitten off or dissolved off with the mouth. The film roll can be advanced manually or by a similar mechanical device as described above where a grippy mechanical finger drags the film to unroll it, or other mechanical means to accomplish the same. The mechanical device may activate by the force and action of opening the lid of such a container. A variation of a roll would be flat pieces that are longer than an individual dose and must be cut, broken, or dissolved off from the rest of the long piece. Such roll containers or long-flat containers provide additional space in comparison with the small flat flip-top prior art containers for promotion, advertising, education, warnings, larger-print ingredient labels, instructions, and items of delight such as movie characters, and it is an aspect of the snacks that the containers for orally soluble edible films be used for these items. A further aspect of the containers is the shape of the container itself. The shape can entertains, delights, educates, warns, instructs, promotes or advertises. For example, the container can be made into the form of a cartoon character or a company symbol. A further aspect is a container which is utilitarian in addition to its containing and dispensing of the film. For example, the container may be part of a keychain or a pants belt or a child's shoe, or be designed to fit within a compartment thereof.

In FIG. 15 an example container 1501 with stacked film 1502 therein is depicted. A sheet of film 1504 may be removed when the container lid 1504 is open.

In FIG. 16, an example container 1601 with stacked film 1602 therein is depicted. The container lid 1603 includes a spring-loaded dispensing finger 1605 for dispensing a piece of film 1604 for consumption.

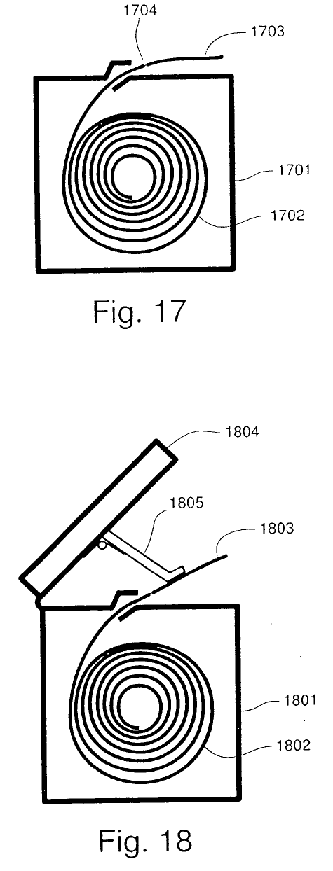

In FIG. 17, an example container 1701 with rolled film 1702 therein is depicted. The film exits the container at a suitable opening and a weakened or perforated portion of the film 1704 is provided for easy removal of a single piece of film 1703 for consumption.

In FIG. 18, an example container 1801 with rolled film 1802 therein is depicted. The container has a lid 1804 that incorporates a spring loaded dispensing finger 1805 that serves to extract a piece of film 1803 from an opening on the container.

Methods for Making Film and Snacks

Methods for making films that can be adapted to making the snacks are documented in the following references each is which is hereby incorporated by references in its entirety: U.S. Pat. Nos. 4,713,243; 5,700,478; 5,948,430; 6,177,096; and 6,284,264; Japanese Patent No. JP-A-05-236885; and U.S. Patent Application Publication Nos.: US 2001/0046511; US 2001/0022964; and US 2002/0131990.

The materials of the desired film are first mixed in liquid form. Solvents may be used to achieve the mixture, such as water, water-dispersible polymers, alcohols, or other solvents.

For example, the films may be cast, molded, extruded, poured or sprayed. A moving belt or drum with or without a backing paper, or any other suitable surface or carrier can be used for receiving the material, hardening/drying or solidifying it, cutting/slitting/shaving/removing it. A drying phase may be employed such as air drying, baking, vacuum drying or dehydrating, such as with circulating warm air. When dry, the film may proceed to a rolling station where it is rolled up for later cutting and packaging.

If a carrier is used, the carrier material should have a surface tension which allows the polymer solution to spread evenly across the intended coating width without soaking in to form a destructive bond between. the two. Examples of suitable materials include non-siliconized polyethylene terephthalate film, non-siliconized kraft paper, polyethylene-impregnated kraft paper, or non-siliconized polyethylene film.

The coating of the solution onto the carrier material can be performed using any conventional coating equipment. A more useful coating technique would involve a knife-over-roll coating head.

The thickness of the resulting film depends on the concentration of solids in the coating solution and on the gap of the coating head and can vary between 5 and 200 .mu.m. Drying of the film can carried out in a high-temperature air-bath using a drying oven, drying tunnel, vacuum drier, or any other suitable drying equipment, which does not adversely affect the active ingredient(s) or flavor of the film.

Some methods for preparing films are capable of encapsulating the oil ingredients (if any) within the film-forming matrix and maintaining the integrity of the film, even when the film contains oils in amounts of 10 wt % or more.

In certain methods for preparing films according to the invention, the film-forming ingredients are mixed and hydrated with water separately from the water-soluble ingredients, which are mixed in aqueous solution separately from the organic ingredients and surfactants. In these methods, the final formulation may be produced by mixing the film-forming phase with the aqueous phase, then mixing in the organic phase, which includes surfactants, such as Polysorbate 80 and Atmos 300. This mass is mixed until emulsified. In other embodiments, the aqueous and film forming phases are combined into a single phase by dissolving the water soluble ingredients in the water and then adding the gums to hydrate. The organic phase is then added to this single aqueous phase.

The resulting formulation is cast on a suitable substrate and dried to form a film. The film may be air-dried or dried under warm air and cut to a desired dimension, packaged and stored. The film can contain from about 0.1% to about 10 wt % moisture, preferably from about 3% to about 8 wt % moisture, even more preferably from about 4 to about 7 wt % moisture.

The film-forming phase can include pullulan and stabilizing agents such as xanthan gum, locust bean gum and carrageenan. These ingredients are mixed and then hydrated in water for about 30 to about 48 hours to form a gel. The water may be heated to a temperature of about 25 to about 45.degree. C. to promote hydration. The amount of water may be about 40 to 80% of the gel. The resulting hydrated gel is then chilled to a temperature of about 20 to about 30.degree. C. for about 1 to about 48 hours. The water may be deionized.

The aqueous phase can include ingredients such as coloring agent(s), copper gluconate and sweetener. The water may be deionized and the amount of water used is about 5 to about 80 wt % of the final gel mixture.

If sodium saccharin and copper gluconate are both ingredients in the formulation, it may be desirable to dissolve them separately in solution to avoid precipitation.

In a useful method of producing essential oil containing films, it is possible to hydrate the film-forming ingredients and combine all of the ingredients without heating. That useful method of producing films comprises dissolving the water-soluble ingredients in water to form an aqueous mixture; mixing the film-forming ingredients in powder form to form a powder mixture; adding the powder mixture to the aqueous mixture to form a hydrated polymer gel; stirring the hydrated polymer at room temperature for about 30 minutes to about 48 hours; mixing the cooling agent, thymol and menthol in the flavor oil to form an oil mixture; adding methyl salicylate; eucalyptol and surfactants to the oil mixture; adding the oil mixture to the hydrated polymer gel and mixing until uniform; deaerating the film until air bubbles are removed, casting the uniform mixture on a suitable substrate; and drying the cast mixture to form a film.

A useful method for making an essential oil containing film hydrates the film-forming ingredients without heating the water. Heating the ingredients increases energy costs in the manufacturing process. Moreover, heating results in undesirable losses of volatile ingredients to evaporation, which also affects the germ killing activity of the composition due to the loss of essential oils. Further, mixing the oils in two steps minimizes the amount of flavor lost.

The film-forming ingredients may be hydrated and mixed without heating due to an ionic effect known as the Donnan equilibrium. Hydrating the film-forming agents in the presence of electrolytes in solution effectively lowers the viscosity of the polymer gel being formed, thus increasing the efficiency of the hydrating process. The water-soluble ingredients of the formulation provide the electrolytes, which are dissolved in the hydration solution prior to addition of the film-forming ingredients. High-shear mixing also accelerates hydration, which delumps the powders, providing greater it surface area for water contact. In addition, local heating effects, generated in the shear regions, provide energy for hydration without substantially raising the temperature of the mass.

Both copper gluconate and saccharin at the same time to the aqueous solution might be avoided, as a precipitate will form. Thus, it is useful to combine sweeteners other than saccharin with copper gluconate.

Dissolving or Disintegrating Snack

Many of the snacks disclosed herein will solvate in the saliva found in the human mouth and dissolve or disintegrate completely within 60 seconds, or within 45 seconds, or within 30 seconds, or within 20 seconds or within 15 seconds or within 10 seconds, or within 5 seconds, or within some other time interval. A snack will be considered to have dissolved or disintegrated completely at such time as it has converted substantially to a syrup or liquid or has broken into particles of such size and mass as to be difficult for the human tongue to detect by size and shape, although the flavor and other sensations of the snack linger. If the snack exhibits the qualities of softness, flexibility and wettability, those qualities should accelerate the time at which the tongue cannot detect the film of the snack.

Mass or Interior Volume

The snacks disclosed herein typically are small sized for placement in a human mouth, and thin to encourage rapid dissolution or disintegration. Films used in the snacks disclosed herein may be of any desired thickness, length and width. An example pliable and easily soluble, edible film may be from less than about 0.0005 inches thick to more than about 0.100 inches thick, such as from about 0.001 to about 0.002 inches thick. Length and width may be chosen so that the snack fits easily on a human tongue with or without folding, such as less than about 1.5 inches in width or length.

In a snack having such dimensions, there is little interior volume and little mass. Low interior volume leaves little room for flavor, sweetener and other ingredients. In addition, use of too much typical candy flavor results in bitterness. As described in greater detail below however, strongly flavored non-bitter snacks have been achieved. Fruit flavored and sour fruit flavored snacks are examples.

Snack Examples:

The following examples illustrate some ways to implement the snacks. The examples are considered to be illustrative only and are in no way limiting. Two example base film formulations are used to which other ingredients are added that make each example film unique. The examples will refer to base film formulations, in which the numbers represent parts by weight:

Base Film Formulation A:

| | |
|---|---|
| 28.74 | Corn starch (film former) |
| 20.92 | Gelatin (film former) |
| 12.55 | Casein (binder, emulsifier, film former) |
| 8.37 | Carrageenan (film former, thickener) |
| 7.32 | Glycerin (plasticizer) |
| 6.28 | Sorbitol (plasticizer) |
| 6.28 | Sorbitan Fatty acid ester (surfactant) |
| 5.75 | Sucrose Fatty acid ester (emulsifier, dispersant) |
| 1.05 | Aspartame (sweetener) |
| 1.05 | Acesulfame K (sweetener) |
| 0.35 | Sucralose (sweetener) |
| 1.36 | Tartaric Acid (saliva enhancer) |
| 100.00 | |

This is a pullulan-free tripartite sweetener film formulation. To this basic formulation is added distilled water (in sufficient quantity that given the other ingredients to be added to this base formulation, the fully hydrated mixture is of the proper viscosity for the type of film making equipment used, for example 1,000 cps. The materials are allowed to hydrate under slow mixing until the ingredients are fully hydrated, then allowed to stand 24 hours under refrigerated conditions.

Base Film Formulation B:

| | |
|---|---|
| 95.29 | Pullulan - PF20 by Hayashibara |
| 2.06 | Atmos 300 (surfactant) |
| 0.55 | Acesulfame potassium (sweetener) |
| 0.55 | Aspartame (sweetener) |
| 0.18 | Sucralose (sweetener) |
| 1.38 | Tartaric acid (saliva enhancer) |
| 100.00 | |

To this is added distilled water (in sufficient quantity that given the other ingredients to be added to this base formulation, the fully hydrated mixture is of the proper viscosity for the type of film making equipment used, for example 1,000 cps. The materials are allowed to hydrate under slow mixing until the ingredients are fully hydrated, then allowed to stand for 24 hours under refrigerated conditions.

Example 1:

5.95 parts of highly-concentrated oil-soluble natural and artificial orange flavoring from Flavors of North America, Carol Stream, IL are added to Base A, along with 11.4 parts of additional citric acid to enhance the flavor and the salivary response, and the mixture is thoroughly blended and made into film on an endless stainless steel belt system with no backing paper. The dried film is spooled into rolls. The film is then fed from the rolls into a printer which uses vegetable based inks to print a company logo and company name repetitively, through an ink dryer, and into an automated cutter/packager which puts discrete pieces of 0.8 inches by 1.2 inch by 0.0018 inch film in stacks of 32 pieces. The flat flip-top container into which the discrete pieces of film are stacked is outfitted with a rubber-coated spring-loaded mechanical finger which activates in conjunction with the lid opening to drag one piece of film from the stack under uniform spring-loaded tension. The film is placed on the tongue of a consumer and provides powerful, non-bitter fresh-orange flavor and a medium degree of tartness. The flavor lasts for more than one minute as the oil-soluble flavor coats the oral cavity and lingers. As the lid is closed, the mechanical finger retracts and lays parallel to the flat box, ready for the next opening of the lid. The printing on the roll of film is situated so that when the film is cut into discrete pieces, each piece has the desired printing of a company logo (graphic) and name (text).

Example 2:

5.84 parts of highly-concentrated oil-soluble artificial grape flavoring from Flavors of North America are added to Base B, along with 11.65 parts of additional citric acid to enhance the flavor and the salivary response, and the mixture is thoroughly blended and made into film on conveyor belt with backing paper. The dried film and backing paper are spooled into rolls. The film and backing paper are then fed from the rolls into a printer which uses vegetable based inks to print a series of color photographs of zoo animals repetitively, through an ink dryer, through a take-up system which separates the film from the backing paper, and into an automated slitter/cutter/packager which creates small rolls of 1.1-inch diameter. When completely unrolled, the piece of film (if not cut or broken off is 0.8 inches wide by 40 inches long by 0.0016 inch. The rounded-box flip-top container into which the discrete pieces of film are stacked is outfitted with a rubber-coated spring-loaded mechanical finger which activates in conjunction with the lid opening to advance the roll of film by a distance of 1.2 inches under uniform spring-loaded tension. The lid is snapped shut which cuts the film at 1.2 inches long. The cut piece of film is placed on the tongue of a consumer and provides powerful, non-bitter grape flavor and a medium degree of tartness. The flavor lasts for more than one minute as the oil-soluble flavor coats the oral cavity and lingers. As the lid is closed, the mechanical finger retracts and lays parallel to the lid of the box, ready for the next opening of the lid. The printing on the original roll of film is situated so that when the film is slit and cut into discrete smaller rolls, each smaller roll has the desired printing of zoo animal photographs.

Example 3:

5.86 parts of highly-concentrated oil-soluble natural and artificial lemon flavoring from Flavors of North America are added to Base A, along with 5.10 parts of additional tartaric acid and 21.9 parts of additional citric acid to promote a sour-lemon flavor and a high salivary response, and the mixture is thoroughly blended and made into film on an endless stainless steel belt system with no backing paper. The dried film is fed directly from the stainless steel belt into a printer which uses inks made of Base A to which spearmint oil has been added at 20% of the ink weight, which prints the brand name of the film product repetitively, then through an ink dryer and into an automated slitter/cutter/packager which creates small rolls of 1.1-inch diameter. When completely unrolled, the piece of film (if not cut or broken off) is 0.8 inches wide by 40 inches long by 0.0016 inch. The rounded-box flip-top container into which the discrete pieces of film are stacked has a hinged lid that snaps open and shut. The film is perforated by the cutting/packaging equipment at 1.2-inch intervals, so that when the film is unrolled, it can be torn off easily. After a piece is torn off, the lid is closed to ensure cleanliness of the remaining pieces. The cut piece of film is placed on the tongue of a consumer and provides powerful, non-bitter lemon flavor and a high degree of sourness/tartness, and a hint of spearmint flavor from the flavored ink. The two flavors lasts for more than one minute as the oil-soluble flavor coats the oral cavity and lingers. The tartness is powerful but begins to fade more quickly than the flavors.

Example 4:

5.86 parts of highly-concentrated oil-soluble natural and artificial raspberry flavoring from Flavors of North America are added to Base A, with the exception that the plasticizers glycerin and Sorbitol are cut in half to make the film slightly more brittle. This is to facilitate the breaking off described below in this paragraph. The mixture is thoroughly blended and made into film on an endless stainless steel belt system with no backing paper. The dried film is fed directly from the stainless steel belt into a printer which uses inks made of Base A to which tartaric acid and citric acid have been added at 10% and 20% of the ink weight respectively, which prints the trade name of the flavor (for example “Raging Razzberry™”) of the film product along with cartoon representations of raspberries repetitively, then through an ink dryer and into an automated slitter/cutter/packager which creates small rolls of 1.1-inch diameter. When completely unrolled, the piece of film (if not broken off) is 0.8 inches wide by 40 inches long by 0.0016 inch. The rounded-box flip-top container into which the discrete pieces of film are stacked has a hinged lid that snaps open and shut. When the lid is opened and the film is partially unrolled, since the film is slightly brittle due to the reduction of plasticizers, it breaks off easily by bending. The user can create a piece as long as desired and is not limited to discrete pieces of size specified by the manufacturer. After a piece is torn off, the lid is closed to ensure cleanliness of the remaining pieces. The broken-off piece of film is placed on the tongue of a consumer and provides powerful, non-bitter raspberry flavor and, due to the acidified ink, a degree of sourness/tartness. The instructions on the package indicate that the printed side should contact the tongue, and the result is that the sourness is tasted even before the raspberry flavoring. The raspberry flavor lasts for more than one minute as the oil-soluble flavor coats the oral cavity and lingers. The tartness begins to fade more quickly than the flavor.

Example 5:

5.86 parts of highly-concentrated oil-soluble artificial cherry flavoring from Flavors of North America are added to Base A, along with 0.5 parts of FD&C red dye and 12.1 parts of citric acid. The mixture is thoroughly blended and made into film on an endless stainless steel belt system with no backing paper. The dried film is spooled into rolls. The film is then fed from the rolls into an automated slitter/cutter/packager which creates small rolls of 1.1-inch diameter. When completely unrolled, the piece of film (if not yet cut) is 0.8 inches wide by 40 inches long by 0.0016 inch. The rounded-box flip-top container into which the discrete pieces of film are stacked has a hinged lid with a metal cutter blade that snaps open and shut. When the lid is opened and the film is partially unrolled, then the lid shut, the cutter blade cuts off a discrete piece of film. The user can create a piece as long as desired and is not limited to discrete pieces of size specified by the manufacturer. After a piece is cut off, the lid is closed to ensure cleanliness of the remaining pieces. The broken-off piece of film is placed on the tongue of a consumer and provides powerful, non-bitter cherry flavor and a medium degree of sourness/tartness. The film is brightly and deeply colored red. The raspberry flavor lasts for more than one minute as the oil-soluble flavor coats the oral cavity and lingers.

Example 6:

5.25 parts of highly-concentrated oil-soluble artificial blueberry flavoring from Flavors of North America are added to Base B, along with 2 parts of additional maleic acid and 1.2 parts of FD&C blue dye, and the mixture is thoroughly blended and made into film on an endless stainless steel belt system with no backing paper. The dried film is spooled into rolls. The film is then fed into an automated cutter/packager which puts discrete pieces of 0.8 inches by 8 inch by 0.002 inch film in stacks of 50 pieces. The flat flip-top container into which the discrete pieces of film are stacked is an elongated box with the hinged side along the 8-inch length. The top is snapped open, and one piece is removed. The consumer can choose to put the end in his/her mouth and continuously dissolve it off, or to break it off and put a smaller piece in the mouth. The film is placed on the tongue of a consumer and provides powerful, non-bitter blueberry flavor and a medium-low degree of tartness. The flavor lasts for more than one minute as the oil-soluble flavor coats the oral cavity and lingers. The film is deeply and brightly colored blue, and after eating an 8-inch long piece of film, the consumers tongue is colored brightly blue.

Example 7:

Two formulations are made. 6.2 parts of highly-concentrated oil-soluble natural and artificial beef flavoring from Flavors of North America are added to Base A, along with 0.15 parts of FD&C dyes formulated to make brown dye, and the mixture is thoroughly blended. 3.3 parts of highly-concentrated oil-soluble artificial green pepper flavoring from Flavors of North America are added to Base A, along with 0.20 parts of FD&C green dye, and the mixture is thoroughly blended. Using a multiple feed stream arrangement, the two differently colored and differently flavored compounds are made into film on an endless stainless steel belt system with no backing paper. The colors/flavors alternate approximately every 0.25 inches, creating a striped effect. The dried film is spooled into rolls. The film is then fed into an automated cutter/packager which puts discrete pieces of 0.8 inches by 8 inch by 0.002 inch film in stacks of 50 pieces. The flat flip-top container into which the discrete pieces of film are stacked is an elongated box with the hinged side along the 8-inch length. The top is snapped open, and one piece is removed. The consumer can choose to put the end in his/her mouth and continuously dissolve it off, or to break it off and put a smaller piece in the mouth. The film is placed on the tongue of a consumer and provides a craving-satisfying flavor combination representing a main course of a dinner. The container being 8 inches long provides sufficient space to print ingredients in a manner large enough to be easily read, in addition to the product identifying and marketing information and graphics, so that a separate blister pack and backing card are not required.

Example 8:

Two formulations are made. 6.2 parts of highly-concentrated oil-soluble natural and artificial beef flavoring from Flavors of North America are added to Base B, along with 0.15 parts of FD&C dyes formulated to make brown dye, and along with 1.1 parts of salt (sodium chloride), and the mixture is thoroughly blended. 3.3 parts of highly-concentrated oil-soluble artificial green pepper flavoring from Flavors of North America are added to Base B, along with 0.20 parts of FD&C green dye, and the mixture is thoroughly blended. The beef-flavored component is made into a film, dried on line, and then the green pepper—flavored component is cast onto top of it, making a two-layer, two-color, two-flavor film. Each layer of film is 0.001 inches thick, and the total film is 0.002 inches thick. The dried film is spooled into rolls. The film is then fed from the rolls into an automated cutter/packager which puts discrete pieces of film in stacks of 32 pieces into flat flip-top containers which have in their peripheral shapes the company logo of the company marketing the films for use by dieters.

Example 9:

6.5 parts of highly-concentrated oil-soluble natural and artificial French fry flavoring from Flavors of North America are added to Base A, along with 0.08 parts of FD&C yellow dye, 2 parts of salt (sodium chloride), and 4.2 parts of soybean oil, and the mixture is thoroughly blended and made into a film. The dried film is spooled into rolls. The film is then fed from the rolls into an automated cutter/packager which puts discrete pieces of film in stacks of 24 pieces into flat flip-top containers. A ring and chain are installed onto each container to make a key chain. The film tastes like French fries, including the satisfaction of a minor amount of fat and salt, satisfying cravings for such foods at a small fraction of the calories, fat, and salt of regular French fries.

Example 10:

4.2 parts of highly-concentrated oil-soluble natural and artificial custard flavoring from Flavors of North America are added to Base B, along with 0.10 parts of FD&C yellow dye, and 2.8 parts of concentrated vanilla extract, and the mixture is thoroughly blended and made into a film. The dried film is spooled into rolls. The film is then fed from the rolls into an automated cutter/packager which puts discrete pieces of film in stacks of 24 pieces into flat flip-top containers. The film tastes like vanilla custard dessert, satisfying cravings for desserts at a small fraction of the calories, fat, and salt of real vanilla custard.

Example 11:

5.3 parts of highly-concentrated oil-soluble artificial butterscotch flavoring from Flavors of North America are added to Base A, along with 0.1 parts of FD&C yellow dye, 0.05 parts of FD&C red dye, 0.02 parts of FD&C green dye, and 3.0 parts of coconut oil, and the mixture is thoroughly blended and made into a film. The dried film is spooled into rolls. The film is then fed from the rolls into an automated cutter/packager which die cuts the film into 16-pointed stars in the caricature of a shining sun and puts sun-shaped, butterscotch-colored pieces of film in stacks of 24 pieces into flat flip-top containers with a circular shape. The film tastes like butterscotch candy, including the satisfaction of a minor amount of fat, satisfying cravings for such candy at a small fraction of the calories and fat as compared to butterscotch hard candies.

Example 12:

5.3 parts of highly-concentrated oil-soluble natural & artificial apple flavoring and 1.4 parts of highly-concentrated oil-soluble artificial cream flavoring both from Flavors of North America are added to Base B, along with 0.3 parts of FD&C green dye, 0.6 parts of cinnamon oil, 0.05 parts of salt (sodium chloride), and 2.2 parts of sunflower oil, and the mixture is thoroughly blended and made into a film. The dried film is spooled into rolls. The film is then fed from the rolls into an automated cutter/packager/embosser which embosses and die cuts the film into a three dimensional apple shape open at the bottom. These pieces are stacked nested 32 deep and put into flat flip-top containers with an apple shape. The film tastes like baked apples with cream and cinnamon, including the satisfaction of a minor amount of fat, satisfying cravings for desserts in a healthy way.

Example 13:

5.8 parts of highly-concentrated oil-soluble artificial popcorn flavoring from Flavors of North America are added to Base A except that no water is used, along with 0.3 parts of FD&C yellow dye and 4.8 parts of peanut oil, and the mixture is thoroughly blended and input to the feed throat of an extruder. A heated die is attached to the extruder which extrudes thin film in a hollow shape with a five-pointed star cross section. The pullulan, being a thermoplastics, lends itself to thermoplastic processes such as extrusion. Immediately downstream from the die a cutter chops the 1 inch diameter star-shaped pieces into 0.25 inch thick pieces. The pieces are dropped into boxes and closed. The film tastes like popcorn, including the satisfaction of a minor amount of fat, satisfying cravings for snacks at a small fraction of the calories and fat as compared to real buttered popcorn. However, it lacks the salt that helps typical popcorn taste good, so it is bland. The three-dimensional shape provides an interesting change from flat films, dissolving on the tongue while providing some texture.

Example 14:

5.8 parts of highly-concentrated oil-soluble artificial popcorn flavoring from Flavors of North America are added to Base A except that no water is used, along with 0.3 parts of FD&C yellow dye and 2.2 parts of salt (sodium chloride), and the mixture is thoroughly blended, made into film, and flat-packed in stacks of 32 rectangular pieces. The film tastes like popcorn, including the satisfaction of a minor amount of salt, satisfying cravings for snacks at a small fraction of the calories and fat as compared to real salted popcorn. However, it lacks the fat that helps typical popcorn taste good, so it is not as satisfying as if fat were added.

Example 15:

Same as Example 14 except that 4.8 parts of peanut oil are added. Now, with both salt and fat added to the popcorn flavor, the film is very satisfying in place of real popcorn which has much higher levels of calories, fat, and salt.

Example 16:

5.5 parts of highly-concentrated oil-soluble artificial spearmint and other mints flavoring from Flavors of North America are added to Base A, along with 0.45 parts of FD&C green dye, and the mixture is thoroughly blended. A chewable candy with L-menthol is made by standard candy methods, then the spearmint film is coated on with pill coating equipment. The resultant pill is put into the mouth and sucked on. The first sensation is a pleasing spearmint candy taste, and then when the film dissolves away, the second sensation (while the spearmint and sweetener are still lingering in the mouth) is the cool breath-freshening effect of the menthol as the inner candy is chewed. Alternatively, biocidal agents can be added as well to prevent bad breath, plaque, gingivitis, and the like.

Example 17:

5.8 parts of highly-concentrated oil-soluble natural & artificial apple flavoring from Flavors of North America are added to Base A, along with 0.23 parts of FD&C green dye, and the mixture is thoroughly blended, the amount of water and gelatin being compatible with capsule forming techniques. The compound is then made into two-part capsules. Separately, a hard candy is made by known methods with sugar and highly-concentrated oil-soluble artificial red hot flavoring from Flavors of North America, then ground into powder. This powder is put into the capsules and the capsule halves joined. The resultant capsule is put into the mouth and sucked on. The first sensation is a pleasing apple candy taste, and then when the film dissolves away, the second sensation (while the cherry and sweetener are still lingering in the mouth) is the red hot spicy effect of the hard candy powder as the powder dissolves in the mouth.

Example 18:

5.3 parts of highly-concentrated oil-soluble artificial cherry flavoring from Flavors of North America are added to Base A, along with 0.08 parts of FD&C red dye, and the mixture is thoroughly blended, the amount of water and gelatin being compatible with gelatin encapsulation techniques. Separately, a mixture is made of soybean oil and numbing ingredients such as are found in the over-the-counter sore-throat numbing spray “Chloraseptic”. The numbing oil mixture is encapsulated into the cherry flavored film with the type of equipment used for vitamin E soft-gels. The resultant soft-gel is put into the mouth and sucked on. The first sensation is a pleasing cherry candy taste, and then when the film dissolves away, the consumer swallows the numbing oil to numb the throat while the pleasant cherry and sweetener taste linger in the mouth. This is more acceptable and pleasant to young children than the gagging effect of a numbing spray.

Example 19:

5.84 parts of highly-concentrated oil-soluble artificial red hot flavoring from Flavors of North America are added to Base A and the mixture is thoroughly blended and made into film by the method of Example 1. When the rectangular piece of film is placed on the tongue and the tongue rubbed against the roof of the mouth, a robust cinnamon-based hot candy flavor is evidenced, lingering for well over a minute. Although there is sufficient citric acid in the Base A to stimulate saliva flow, no tartness is noted in the film taste.

Example 20:

5.95 parts of highly-concentrated oil-soluble natural and artificial orange flavoring from Flavors of North America, along with 5.0 parts tartaric acid and 20.0 parts citric acid are added to Base B and the mixture is thoroughly blended and made into film by the method of Example 1. When the film is ingested in the oral cavity, a powerfully sour/tart fresh-orange flavor is evidenced, with the orange flavor lingering longer than the sourness. Since Base B has pullulan instead of starch, the low pH only has a minor effect on the film, causing slight brittleness.

Example 21:

5.84 parts of highly-concentrated oil-soluble artificial grape flavoring from Flavors of North America are added to Base A and the mixture is thoroughly blended and made into film by the method of Example 1. As the film is in a still wet but nearly dried out state on its way down the stainless steel belt, citric acid powder diluted with maltodextrin is sprinkled on the surface and adheres in the tacky surface of the film without dissolving to any great extent. When the film is ingested in the oral cavity, a powerfully sour/tart grape flavor is evidenced, with the sourness experienced first-(if the film is placed on the tongue acid-side first), then the grape flavor being experienced and lingering longer than the sourness. Because the citric acid is on the film rather than in it, the physical properties of the film are not affected adversely as they would be if the starch-based film was very low pH due to the inclusion of this much acid.

Example 22:

Two formulations are made. 5.82 parts of highly-concentrated oil-soluble natural and artificial apple flavoring from Flavors of North America are added to Base A, along with 0.3 parts of FD&C green dye, and the mixture is thoroughly blended. 3.3 parts of highly-concentrated oil-soluble natural and artificial cranberry flavoring from Flavors of North America are added to Base A, along with 0.17 parts of FD&C red dye and 0.12 parts of FD&C blue dye, and the mixture is thoroughly blended. Films are separately made from each formulation. The films are then adhered to one another with a mixture of binder (such as pullulan) and citric and tartaric acids sufficient to produce the level of tartness as in example 21. The cran-apple flavor is exceptionally sour, which is pleasing to many people, especially children. Because the citric acid is between the film layers rather than in it, the physical properties of the film layers are not affected adversely as they would be if the starch-based film was very low pH due to the inclusion of this much acid.

Example 23:

5.72 parts of highly-concentrated oil-soluble artificial fresh watermelon flavoring from Flavors of North America are added to Base B and the mixture is thoroughly blended and made into film by the method of Example 21, with the blend of citric acid and maltodextrin being replaced by a blend of two microencapsulated acids. One of the microencapsulated acids (145-72L Citric Acid from Loders Croklaan company of Channahon, Ill.) causes a delay of a few seconds before the sourness of the acid is tasted, and the second microencapsulated acid (150-80VS Citric Acid from Loders Croklaan company) causes a longer delay. Because of the variability of the thickness of the encapsulation, the sourness lasts a long time, even over one minute, so that the flavor and the sourness stay together unlike earlier stated examples of sour films.

Example 24:

Pressurized-gas candy powder as described earlier, made with a spearmint flavor, is put into an envelope or pouch made of a cherry-flavored film based on Base B and heat-sealed shut, taking advantage of the thermoplastic nature of the pullulan. The consumer gets a two-part flavor experience, with the flavors being different in timing, and then as the pressure is released from the candy powder, gets a noisy crackling effervescent experience.

Example 25:

5.84 parts of highly-concentrated oil-soluble artificial grape flavoring from Flavors of North America are added to Base A and the mixture is thoroughly blended and made into film by the method of Example 1. As the film is in a still wet but nearly dried out state on its way down the stainless steel belt, grape-flavored pressurized-gas candy powder is sprinkled on the surface and adheres in the tacky surface of the film without dissolving to any great extent. When the film is ingested in the oral cavity, a grape candy flavor is evidenced along with noisy crackling effervescence.

Example 26:

Same as Example 22 except that the citric and tartaric acids between layers is replaced by apple-flavored powdered pressurized-gas candy. Instead of a sour cran-apple, a noisy crackling effervescent sweet cran-apple is experienced.

Example 27:

A two-layer film is made with complementary flavors in each layer. One layer is high in citric acid, and the other layer is high in sodium bicarbonate. They are adhered together with a binder. When dissolved by saliva in the mouth, the acid and the sodium bicarbonate combine and the reaction produces carbon dioxide gas. A pleasing, mouth-filling, mouth-coating effervescence results.

### Vitamin-Containing Orally Soluble Films

Orally soluble food products such as flavor films or other orally soluble food products may be enhanced by the inclusion of nutraceuticals (such as, by way of example, vitamins, minerals, herbs, etc.) in wonderful tasting flavor films or other shapes. Nutraceutical films such as these may release nutraceuticals into the body's various digestive systems much sooner than tablets or capsules. Further, nutraceuticals in flavor films may be much more palatable than in other delivery systems because of the exceptionally great taste that may be incorporated into such films. A particular range of film size and thickness may in some instances be desirable when incorporating nutraceuticals into films. Film formulations can be modified to achieve desired mouth feel and dissolution with larger and/or thicker films.

Vitamins are organic substances that are useful for health, growth, repair, reproduction, healing and maintenance. They are ordinarily included in the diet but some individuals may choose to supplement their vitamin intake. Some example vitamins that may be used in the films herein include the following:

A. Vitamin A (palmitate)—night vision, body growth and tooth development, combat infection, cofactor for various syntheses.

B. Vitamin D (cholecaliferol)—increases calcium absorption, promotes growth and mineralization of bones, promotes sound teeth, enhances phosphorous metabolism, helps maintain citrate levels.

C. Vitamin E (alpha tocopheryl)—antioxidant for fat metabolism, a factory for proper red blood cells, used in cellular respiration and DNA synthesis.

D. Vitamin C (ascorbic acid)—aids in maintenance of collagen (wound and burn healing), enhances iron absorption, used in amino acid metabolism.

E. Vitamin B1 (thiamine)—coenzyme in energy metabolism, nerve maintenance, regulates appetite.

F. Vitamin B2 (riboflavin)—metabolically involved in oxidation/reduction energy production, cofactor with B6 in formation of niacin.

G. Vitamin B3 (niacin)—used in normal growth, reduces cholesterol, used in synthesis of fatty acids.

H. Vitamin B6 (pyridoxine)—involved in nitrogen metabolism, carbohydrate and fat metabolism.

I. Vitamin B12 (cyanocobalamin)—involved in cellular physiological roles (bone marrow and nerve tissue), red blood cell formulation and control of anemia.

J. Folic Acid—involved in the formation of purines and pyrimidines, RNA and DNA.

Some orally soluble films may be thin (such as 40-50 microns) and small (such as 20-35 by 20-35 mm) in order to have an acceptable mouth feel and to dissolve sufficiently quickly. An entire oral strip of this size is of less total weight than many commercially available vitamin tablets. Therefore it could be difficult to fit a daily dose of vitamins into an orally soluble film. However, the size and mass of the orally soluble film can be increased to carry a useful or effective amount of nutraceuticals. Examples of such dimensions are as follow:

Example film dimensions:

| Orally soluble | Orally soluble | Orally soluble film |
|---|---|---|
| film width | film length | thickness |
| 20-80 mm | 20-80 mm | >100 microns |
| 25-35 mm | 35-50 mm | >120 microns |
| 28 mm | 43 | 140 |
| 30 mm diameter | (round) | >150 |
| 20 mm | 40 mm (elliptical) | >80 |
| 10 mm | 80 mm | <200 |

In addition to the dimensions of the orally soluble films, the ingredients are a factor to consider. Example ingredients are found below. Film ingredients can include film base, sweetener, acid/tartness/sour ingredient, colorant, flavoring and nutraceuticals. These ingredients can be included in a variety of combinations, such as the following:

| Example | | | Acid/ | | | |
|---|---|---|---|---|---|---|
| (all | | | Tartness/ | | | |
| given by | Film | Sweet- | Sour | Col- | Fla- | |
| weight | base | ener | ingredient | orant | voring | Nutraceutical |
| #1 | 50 | 0-30% | 0-10% | <10% | <50% | <50% |
| #2 | 60 | 0-50% | 0-30% | <10% | <40% | <50% |
| #3 | 40-60% | 0-50% | 0-40% | <10% | <40% | <60% |
| #4 | 30-70% | 0-50% | 0-50% | <10% | <70% | <70% |
| #5 | 20-80% | 0-50% | 0-50% | <10% | <80% | <80% |
| #6 | 10-90% | 0-60% | 0-50% | <20% | <80% | <90% |

Example film bases are described in greater detail below. Any combination of the above could be used, as well as variations of the above and substitutions for the above. Some example orally dissolvable nutraceutical strips are described in greater detail below. All are listed in parts by weight.

Example 1 (mg in one nutraceutical flavor strip):

| | |
|---|---|
| Harmony Concepts Nutrients Lot | 120.0 |
| #250403-50 | |
| Film base other than water | 60.4 |
| Sucralose | 1.4 |
| Tartaric Acid 2307 | 27.2 |
| FONA EZ 404.884 Orange Flavor | 7.4 |

In Example 1, “Harmony Concepts Nutrients Lot #250403-50” is a vitamin and mineral mixture made by Harmony Concepts of Ogden, Utah, 120 mg of which resembles the formulation found in a daily dose (one tablet) of Flintstones Chewable Tablets Multivitamin Supplement, and in addition includes a minor amount of calcium, as follows:

| | | |
|---|---|---|
| Vitamin A | 2500 | iu |
| Vitamin C | 60 | mg |
| Vitamin D | 400 | iu |
| Vitamin E | 10 | iu |
| Vitamin B1 | 1.05 | mg |
| Vitamin B2 | 1.2 | mg |
| Niacin | 13.5 | mg |
| Vitamin B6 | 1.05 | mg |
| Folic Acid | 300 | mcg |
| Vitamin B12 | 4.5 | mcg |
| Calcium | 7.5 | mg |

In Example 1, “Film Base other than water” (most of the water is dried out during the film making process) indicates the film base marketed by Ascona Ingredients of Mississauga, Ontario, Canada for the product marketed by Jakks Pacific of Malibu, Calif. as “Tongue Tape”. This film base includes:

corn starch

gelatin

water

glycerin

sodium alginate

sorbitol

sorbitan acid ester

Any of these ingredients may be omitted or others substituted in a film base. Other suitable bases and tapes are disclosed elsewhere herein.

An example of the non-water portion of a film base is the following:

| | | More | |
|---|---|---|---|
| | | specific | |
| | Example | example | |
| | Weight | Weight | |
| Ingredient | % Range | % | Specifications |
| Gelatin | 40% to | About | Example Bloom from 150-275 |
| | 85% | 65% | (Can also use lower or higher |
| | | | bloom strength) |
| Starch | 2% to | About | Example tapioca starch, also |
| | 45% | 25% | modified corn/potato starch |
| Alginate | 5% to | About | Example Sodium alginate/propylene |
| | 15% | 5% | glycol alginate |
| Carrageenan | 2% to 5% | About | Kappa or Lambda-alcohol |
| | | 5% | precipitated grade used in tests |

These ingredients can be adjusted to vary the properties of the film, the desired film being exceptionally pliable and fast dissolving. As in standard water-soluble film making, the ingredients are fully hydrated and mixed, spread into a thin sheet, and most of the water is dried out to make the film.

In Example 1, “Sucralose” is an artificial sweetener, tartaric acid provides tartness, flavor enhancement, and masking, and “FONA EZ 404.884 Orange Flavor” is a concentrated oil-based orange flavor by Flavors of North America of Carol Stream, Ill.

In the past although some effort was made to construct a film candy, such films would be of insufficient size to contain the Example 1 nutraceutical formulation, and if made of sufficient size to contain it, would according to their own teaching be too stiff and rigid and would be uncomfortable in the mouth, and would take longer to dissolve away. Further past candy films would not have the excellent flavor of this film, it being based on the concentrated oil-based flavors and other features as disclosed in my previous submittals. The excellent flavor is important since some of the vitamins and/or minerals can have an unpleasant taste or aftertaste if they are not well masked by pleasant flavors, acids, sweeteners, etc.

Another example nutraceutical film or vitamin tape for children is shown below. This orally soluble film containing nutraceuticals is intended for human consumption to maintain or improve a persons physical state or health.

| Nutrient | Dosage per 2 film strips (approx.) | |
|---|---|---|
| Vitamin B1 | 1.05 | mg |
| Vitamin B2 | 1.20 | mg |
| Vitamin B3 | 13.5 | mg |
| Vitamin B6 | 1.05 | mg |
| Vitamin B12 | 4.5 | mcg |
| Folic Acid | 300 | mcg |
| Vitamin C | 60 | mg |
| Vitamin A | 2500 | iu |
| Vitamin E | 10 | iu |
| Vitamin D3 | 400 | iu |

| | |
|---|---|
| Others | As desired. |

An optional aspect of the orally dissolvable films herein is to use microencapsulated vitamins, minerals, and/or herbs, etc., so that their undesirable flavor is not released until in the stomach or intestines. This can be particularly advantageous for the ingredients which taste bad, but is not necessary for all the nutraceutical ingredients. Another optional aspect of the orally dissolvable films is to use a tailored or targeted flavor masking agent, as are known in the flavoring industry, for each bad-tasting ingredient. But even without the further improvement of microencapsulation or special maskers, the Example 1 formula tastes good due to the extraordinary goodness of the films of my prior submittals into which it is placed. Further, the film is very pliable and acceptable in mouth feel, even though it is thicker, and this also helps the larger size be acceptable. Further, it dissolves rapidly even though it is thicker than prior-taught films.

Another example of the films herein, Example 2, illustrates the inclusion of a single nutraceutical, Vitamin C, in large amounts, in the same film base as Example 1 but with grape flavoring instead of orange, and with an equal-sweetness mixture of three sweeteners, Aspartame, Sucralose, and Acesulfame Potassium, instead of.just one or two sweeteners such as Sucralose or Aspartame/Acesulfame Potassium. The combination of three sweeteners results in less, if any, of the aftertaste associated with artificial sweeteners. The Ascona film base is able to take substantial amounts of acid without degradation as in prior film bases, and since Vitamin C is in the form of ascorbic acid, this allows a considerable amount of Vitamin C to be used in a single film. The film can be of ‘normal’ size and thickness for an oral strip (see above), or can be larger and thicker as in Example 1, in which case it can contain substantial amounts of Vitamin C which are immediately available to be absorbed by the body upon dissolution of the film. In Example 2, 120 mg of Vitamin C are contained in a film the size of the film in Example 1 and the tartaric acid is reduced to account for the acidity of the ascorbic acid Vitamin C. In this case the flavor is excellent, since the grape film (see my prior submittals) is already excellent, and ascorbic acid does not have a bad taste which cannot be masked by the sweeteners, flavor, and acidulant in Example 2. A good grape flavor is from Flavors of North America, designated FONA EZ 193.856 Grape Flavor.

Any nutraceutical, and any film base, and any sweeteners, flavorants and acids, may be used in the films herein provided each film piece is large enough and thick enough to contain the nutraceutical while maintaining film cohesiveness, and has comfortable mouth feel and good dissolution.

An optional aspect of the films herein is printing, stamping, spraying or casting information or graphics on the nutraceutical-containing strip. This can be important so that the user does not confuse it with similar candy strips and eat more than the recommended dosage.

Other aspects of the films herein include type of packaging, levels of various ingredients, tongue staining, types and combinations of flavorings/acids/sweeteners, number of film forming agents, multiple colors/flavors/layers, shapes, glow in dark, effervescence, encapsulation, container utility, etc, in addition to containing nutraceutical(s) in, on, or between layers of, the films, but for brevity all of these are incorporated by reference above.

### Medicine-Containing Orally Soluble Edible Films

Orally soluble films can be manufactured with the inclusion of medications (of any type, such as pharmaceuticals, over-the-counter remedies or medicines, antacids, homeopathic remedies, herbal remedies, folk remedies, antimicrobials, etc.) in wonderful tasting flavor films. In such a film, medication is released into the body's various digestive systems much sooner than for example tablets or capsules. Medications in my films are much more palatable than in other delivery systems because of the exceptionally great taste of my films in comparison with prior films. Film size and thickness are also a point of divergence from other medication delivery systems. For example, films with medications greater than 30 mg per dose can be integrated into films, but would suggest modification of the formulation to achieve proper mouth feel and dissolution with larger/thicker films. The disclosure of the orally dissolvable films containing nutraceuticals above is hereby incorporated by reference and is considered applicable to the medicine-containing orally soluble films.

Very thin films may not be able to carry a pharmaceutically effective dose of a desired medication. Therefore the film will be sized to carry a desired amount of medication. Example film dimensions were already given above. Another medication-containing good-tasting film which is large enough and thick enough to contain in two film strips the amount of acetaminophen in a regular-strength tablet (such as Regular-Strength Tylenol), or in three film strips about the amount in an extra-strength tablet is:

28 mm wide

43 mm long

155 microns thick

Example 3 (mg in one medication flavor strip):

| | |
|---|---|
| Acetaminophen | 162.5 |
| Film base other than water | 60.4 |
| Sucralose | 0.47 |
| Aspartame | 1.4 |
| Acesulfame Potassium | 1.4 |
| Tartaric Acid 2307 | 27.2 |
| FONA EZ 518.906 Raspberry Flavor | 7.4 |

Example film bases are also taught above and that teaching is relied upon herein. In Example 3, “Sucralose”, “aspartame”, and “Acesulfame potassium” are artificial sweeteners, tartaric acid provides tartness, flavor enhancement, and masking, and “FONA EZ 518.906 Raspberry Flavor” is a concentrated oil-based raspberry flavor by Flavors of North America of Carol Stream, Ill.

Films of insufficient size to contain the Example 3 medical formulation may not be successful in carrying medications. Likewise, films that are too stiff and rigid and would be uncomfortable in the mouth, and would take longer to dissolve away. Further films using concentrated oil-based flavorings may have the ability to mask medicine flavors well. An optional aspect of the films herein is to use microencapsulated medications, so that their undesirable flavor is not released until in the stomach or intestines. This can be used if the ingredients which taste bad, but is not necessary for all the medication ingredients. Another optional aspect of the films is to use a tailored flavor masking agent, as are known in the flavoring industry, for each bad-tasting ingredient. But even without the further improvement of microencapsulation or special maskers, the Example 1 formula is much improved from prior medicated films due to the extraordinary goodness of the films herein. Further, the film may be very pliable and acceptable in mouth feel, even though it is thicker, and this also helps the larger size be acceptable. Further, the film may dissolve rapidly.

Any medication, and any film base, and any sweeteners, flavorants and acids, may be used in the films provided each film piece is large enough and thick enough to contain the medication while maintaining film cohesiveness, and has comfortable mouth feel and good dissolution.

Other optional aspects of the films are as already given above.

### Electrolvte-Containing Orally Soluble Edible Films

The disclosure above is hereby incorporated by reference.

Electrolytes and other elements are lost to the body during sweating such as during rigorous exercise. It is desirable to have a technique for replacing those electrolytes and other elements. There are commercial drinks such as Gatorade which accomplish this, but they have the disadvantage of being bulky and heavy and at times inconvenient. Orally-dissolvable films containing electrolytes can be used to replace electrolytes and other elements lost in the body during sweating. For example, a runner can stop where water is available and ingest some electrolyte-containing orally soluble edible film and ingest some water to enjoy electrolyte and lost element replacement.

Such films can include all of the electrolytes and elements lost during exercise, or some or all of the ingredients of nutritional or electrolyte replacement drinks such as Gatorade. The films, if desired, can also be used to provide a source of carbohydrates if it is felt that the replacement of electrolytes without replacement of carbohydrates is less desirable. In addition, the relatively thin film sheets herein can be produced more economically and shipped more economically than a bottle drink, making a potentially lower cost product for the consumer. The consumer could make use of the film sheets herein by dissolving them on the tongue, with or without with or without then drinking water. The orally dissolvable films can be realized in such films by incorporating all the desired electrolyte ingredients into a thin flexible film.

For example, the following ingredients (weight in grams) can be made into a flexible water-soluble film:

| | |
|---|---|
| Pullulan PF-20 | 0.100 |
| FONA 856.193 Grape Flavor | 0.020 |
| Acesulfame Potassium | 0.006 |
| Aspartame | 0.006 |
| Sucralose | 0.002 |
| Citric Acid | 0.030 |
| Sodium Chloride | 0.080 |
| sodium citrate | 0.030 |
| mono-potassium phosphate | 0.030 |

The film is simply placed on the tongue and allowed to dissolve. A human mouth will ordinarily produce additional saliva to assist in dissolving the film after the film contacts the tongue. An athlete can place the film in his mouth while exercising or in a game. The film may be made sugar and carbohydrate free so as to not promote tooth decay or add to a dieter's daily ration of calories.

The various enhancements and variations of the orally soluble film concepts mentioned herein may also be applied to electrolyte-containing films.

### Delivery Units of Thick Orally Soluble Polymer

Orally soluble films for snacks, nutraceuticals, medicines, electrolyte replacement, mouthwash ingredients (including without limitation mints, essential oils, antimicrobials, etc.) may be thin for quick dissolution in the oral cavity. Even the “thicker” nutraceutical and medicinal films of the above are in typical applications less than 200 microns thick.

If desired, a much thicker delivery unit (for delivery of anything including without limitation flavor, sweetened flavor, sweetened acidified flavor, medicine, electrolytes, mouthwash ingredients, nutraceuticals, fats, salt, and/or color) comprising water soluble polymers may be assembled. The much thicker delivery unit may be made to stick to the roof of the mouth or the gum line or other oral tissues, but can also be made to be free floating. It can be made to have acceptable mouth feel either by making the polymer highly flexible or by making it semi-flexible and having the length and width be much smaller than a typical orally soluble film. Flexibility can be obtained by means of higher moisture content, or higher plasticizer content, or by use of more flexible polymers or combinations of polymers. Optional ingredients are described elsewhere herein.

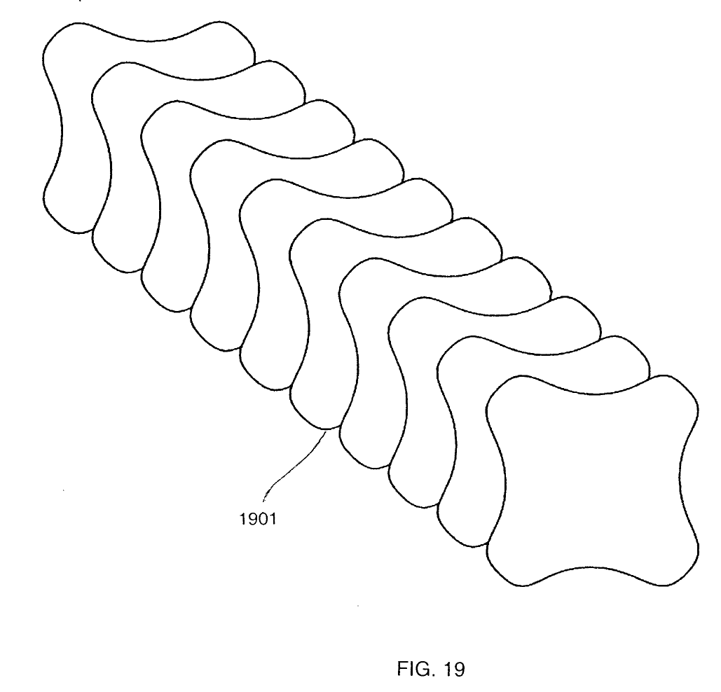

The key in this example is to make the orally soluble delivery unit much thicker than the other examples in this document. For example a snack-type flavor delivery system of 800-1,200 microns thick could be employed, with irregular outer dimensions of about 13 mm by 13 mm. The four-pointed star-shaped breath-mint snack shown in FIG. 19 as reference numeral 1901 is in a multi-pack configuration. The radial dimensions of the orally soluble delivery unit will be limited by the size and comfort parameters of the human mouth, but generally it is expected that the orally soluble delivery unit will have a radial dimension of less than 500 mm, or less than 300 mm, or less than 200 mm, 100 mm, or less than 80 mm, or less than 60 mm, or less than 50 mm, or less than 40 mm or less than 20 mm or less than 10 mm. The orally soluble delivery unit in this example as well as throughout this document, can be made in any known geometric shape, including round, square, rectangular, triangular, spherical, pentagonal, n-sided polygonal where n is an integer, curved, or according to any other desired shape. The thickness of the orally soluble delivery unit in this example may be more than 200 microns, more than 400 microns, more than 500 microns, more than 700 microns, more than 800 microns, more than 1000 microns, more than 1200 microns, more than 1500 microns or more than 2000 microns.

The orally soluble delivery unit product may be placed on the tongue so that the tongue elevates it to the roof of the mouth. It sticks better to the roof of the mouth than to the tongue, and so is transferred to the roof of the mouth. The consumer rubs the tongue against the example polymeric breath mint, which contains for example spearmint oil, menthol, artificial sweeteners, a minor amount of acid for saliva enhancement, and Pullulan or gelatin as the polymer. The delivery unit is so thick that it lasts for several minutes, for example 5-10 minutes, depending on the amount of saliva and the vigor of the rubbing tongue. This is compared to 5-30 seconds for typical orally soluble delivery units such as Listerine Pocket Paks Strips by Warner Lambert, or such as Tongue Tape by Jakks Pacific. This longer dissolution time means the flavor (or medicine, or nutraceutical, or mouthwash ingredients, or etc.) is in the oral cavity for much longer, which can be desirable in many cases. Yet, it is not bothersome as for example a hard-candy breath mint, which rattles around in the mouth and can be embarrassing to the consumer, because the delivery system of this invention is preferably firmly adhered to the roof of the mouth. This allows free speech and little chance of detection, all during the many minutes of effectiveness. Even if a non-adhering formulation is used for the delivery unit, the polymers are less noisy than a sugar-candy mint when contacting the teeth. The orally soluble delivery unit may also be used to make a breath freshener, mint or snack (such as by using oil-based flavors) by using other ingredients mentioned elsewhere herein. Because the delivery system of this example has so much more volume than typical orally soluble films, much more active ingredient (flavor, acid, sweetener, medicine, nutraceutical, mouthwash ingredients, etc.) can be put into a single unit dose.

Pullulan is an excellent candidate for the orally soluble polymer because it an excellent adhesive. Gelatin is also an excellent polymer candidate. Mixtures of polymers can also be effective, for example the mixture of starch, gelatin, and alginate (all film forming polymers) in the film base marketed by Ascona Ingredients of Mississauga, Ontario, Canada for the product marketed by Jakks Pacific of Malibu, Calif. as “Tongue Tape”. Appropriate base films are disclosed elsewhere herein. The base film ingredients can be adjusted to vary the properties of the delivery unit, the desired delivery unit being exceptionally pliable to semi-rigid, and fast dissolving to slow dissolving, depending on the application. Increases in any or all of glycerin, sorbitol, or sorbitan acid ester might be considered to make the film more pliable. The manufacturing method can be the same as in standard water-soluble film making, the ingredients are fully hydrated and mixed, spread into a sheet, and most of the water is dried out to make the film. However, in this case, either very slow drying or making the film in multiple layers and drying each layer individually before spreading the next layer is advisable to avoid wrinkles. The film can then be cut by slitting, die-cutting, or any other cutting method which gives good results.

Examples 4-12 show alternative formulations for the orally soluble delivery devices, with weight in grams. This is the non-water portion (in most cases, little water is left after drying the product).

| | Example #: | | | | | | | | |
|---|---|---|---|---|---|---|---|---|---|
| | 4 | 5 | 6 | 7 | 8 | 9 | 10 | 11 | 12 |
| Hydroxypropylmethylcellulose | 28 | 20 | | 20 | | | | | |
| Pullulan | | | | | | | | 113 | 113 |
| Gelatin 275 | | | 20 | | 20 | | 6.0 | | |
| Gelatin 0 | | | | | | 10.0 | | | |
| Pectin | | 8.3 | 8.3 | 8.3 | 8.3 | 18.3 | 4.0 | 28.3 | 28.3 |
| Gum Arabic | | | | | | | 18.3 | | |
| Glycerin | 2.4 | | | 1.2 | 1.2 | 1.2 | 1.2 | | |
| Acesulfame Potassium | | | | | | | | | 1.1 |
| Aspartame | | | | | | | | | 1.1 |
| Sucralose | 0.7 | 0.7 | 0.7 | 0.7 | 0.7 | 0.7 | 0.7 | 3.3 | 0.4 |
| Citric Acid | | | | | | | | | 1.2 |
| Tartaric Acid | 13 | 12.8 | 12.8 | 12.8 | 12.8 | 12.8 | 12.8 | 63.9 | |
| FONA EZ 404.884 Orange Flavor | 3.5 | 3.5 | 3.5 | 3.5 | 3.5 | 3.5 | 3.5 | 17.3 | |
| FONA EZ 064.932/EN POWDER | | | | | | | | | 24.5 |
| Spearmint | | | | | | | | | |
| Total | 48 | 45 | 45 | 46 | 46 | 46 | 46 | 226 | 170 |

### Rapidly Dissolving Chewable Tablets

Another orally soluble delivery system is a rapidly dissolving chewable tablet. These tablets can be quite solid, but can be chewed and when chewed disappear quickly, in as little as 1-4 chews or more. The tablets are made with a matrix comprising one or more rapidly dissolving or at least rapidly disbursing or disintegrating materials, for example a directly compressible soluble excipient by JRS Pharma called Emdex. Aiding the dissolution of the tablet into the saliva upon chewing are one or more disintegrants (and so-called superdisintegrants), such as Explotab (a swelling superdisintegrant by JS Pharma), Actisol (a capillary action

superdisintegrant by FMC Corp), and/or Primelose (an elastic recovery superdisintegrant by Avebe). I have found that combinations of superdisintegrants are very effective. The following Examples 13 and 14 illustrate candy-type rapidly dissolvable chewable tablets:

Example
(mg per tablet):

| | 13 | 14 |
|---|---|---|
| Emdex | 677.4 | 636.5 |
| Primelose | 14.9 | 14.0 |
| Pullulan | 7.5 | 7.0 |
| Explotab | 29.8 | 28.0 |
| Acesulfame Potassium | 1.9 | |
| Aspartame | 1.9 | |
| Sucralose | 0.6 | 1.2 |
| Citric Acid | 2.2 | 12.7 |
| Tartaric Acid | | 11.4 |
| FONA EZ 064.932/ENSpearmint Flavor | 43.7 | |
| FONA EZ 884.806/EN Orange Flavor | | 69.3 |
| Total: | 780 | 780 |

These rapidly dissolving tablets can be carriers for candy and other confections, medicines, nutraceuticals, mouthwash-type ingredients, or any other item desired to be released into the mouth quickly.

### Extruded Foods and Processes for Making the Same

These compositions and processes may be applied to food products produced using any type of extruder, including cooking extruders. In such applications, flavors are often incorporated into food products prior to their exiting the extruder. Examples of such food products, without limitation, include breakfast cereals, snack foods, pet foods, and agricultural animal foods.

Many if not most of the subject food products are produced in shaped forms, flakes or pellets that incorporate voids to improve the texture and feel of the food product in the mouth, as well as its feel, appearance, perceived value, chewability, digestibility and other characteristics. These voids are typically produced by expansion of steam or compressed gasses incorporated under pressure during travel of the ingredients through the extruder. The expansion typically takes place with the ingredients at high temperature as well. The high temperature is necessary for steam expansion, and high temperatures are typically used to cook the food product and to initiate a chemical reaction to release gases or vapors into the ingredients.

Flavors incorporated into the subject food products can be flashed off (evaporated) during the rapid expansion at the end of the extrusion process when the pressure on the ingredient/steam/compressed-gas mixture is released under high pressure at the end of the extruder. This loss of flavor prevents internal flavoring in many subject food products that must then be subsequently coated with flavoring to achieve an acceptable palatability. Many of the subject food products would be improved if the flavors were incorporated throughout the product. Incorporating the flavors at the extruder rather than applying them as a coating to the food product after extrusion is complete would also lower the cost of many of the subject food properties.

A technique is described to increase the percentage or amount of flavors (or other desirable volatile components) retained in an edible product (food, medicine, nutraceutical, remedy, or any other) following extrusion as compared to prior to extrusion. This technique may or may not obviate the need to coat extruded food with a flavor coating following extrusion.

The technique can involve the following steps.

A. Use an extruder with a controllable restriction at its outlet in order to maintain internal pressure at the outlet of the extruder at any desired level set by the operator. Such a restriction could include, by way of example and without limitation, a gear pump or an electromechanical valve, or any other mechanical restriction. The restriction will aid in keeping pressure within the extruder constant to avoid flashing off flavorings.

B. Set the temperature profile along the length of the barrel of the extruder so that the food ingredients input into the extruder reach the necessary temperatures for cooking, mixing, or other such functions desired in making a food product before reaching the outlet of the extruder, and are also then are cooled to a desired cooler temperature less than the peak temperature that the ingredients reach within the extruder prior to reaching the restriction at the extruder outlet. This cooling is intended to assist in reducing flavor loss upon expansion of the food product at the outlet of the extruder.

C. Inject a gas (or a liquid that upon heating will become a gas) into the food ingredients at an appropriate place within the extruder so that the gas and the ingredients are pressurized as the mixture moves toward the restriction at the outlet of the extruder. Examples of gases that can be gas used include, without limitation, include carbon dioxide, nitrogen, argon, steam, mixtures of gases, and mixtures of steam and gases. The location for injection of the gas will vary with extruder design, but can be anywhere along the length of the extruder downstream of the point where the ingredients have formed a gas seal with the bore of the extruder that will prevent blowback of the injected gas through the inlet of the extruder. In lieu of injecting gas into the extruder, gas generating chemicals can be incorporated into the ingredients and gas can be generated during the heating of the ingredients in the extruder, or water in the ingredients can be converted to steam during the heating of the ingredients in the extruder.

D. Include desired flavorings in the food ingredients that are input into the extruder through its inlet, provided the temperature of the food ingredients within the extruder do not reach the volatilization temperatures of such flavorings prior to forming the above described gas seal. A caveat is that although this may preserve the volatile flavor through use of a gas seal, the flavor may still be degraded from the relatively high temperatures that the food product ingredients reach within the extruder. Alternatively, the desired flavorings may be injected into the food ingredients within the extruder downstream of the plug, such as at a point where the ingredients have been cooled below the temperature which would degrade the flavorings. The injection point can be a sufficient distance upstream from the restriction described above that the flavors will be sufficiently distributed into the mixture of ingredients by the action of the extruder to achieve relatively uniform flavoring before the ingredients the restriction at the outlet of the extruder.

Use of the outlet restriction, gas injection or gas generation, cooler temperature flavor injection, and/or an appropriate temperature profile, as described above, allow the ingredients to leave the extruder at an appropriate lower temperature and at a desired relatively high pressure while preserving flavoring within the food product. The lower temperature helps to avoid flavor degradation and helps to retain a higher percentage of flavors that may otherwise flash off (volatilize, evaporate) at higher temperatures. The higher pressure produces the desired voids within the subject food products even though the ingredients exit at a lower temperature. Later coating of the food product with a flavoring may be avoided or included as desired.

### Generally

While edibles, their compositions and manufacturing methods and containers have been described and illustrated in conjunction with a number of specific ingredients, materials and configurations herein, those skilled in the art will appreciate that variations and modifications may be made without departing from the principles herein illustrated, described, and claimed. The present invention, as defined by the appended claims, may be embodied in other specific forms without departing from its spirit or essential characteristics. The configurations of snacks described herein are to be considered in all respects as only illustrative, and not restrictive. All changes which come within the meaning and range of equivalency of the claims are to be embraced within their scope.

1. A medicine-containing orally soluble edible film comprising:
one or more layers of film,
said film being orally soluble such that it disintegrates quickly upon placement in a human mouth;
at least one flavoring present in said film, said flavoring providing a strong flavor sensation to a person who places the snack on his or her tongue,
and a dosage of a desired medicine or medicament intended to be useful to the functioning of the human body, such as in times of sickness;
wherein said flavoring serves at least in part to mask flavor of said medicine or medicament.

2. A film as recited in claim 1 wherein said medicine or medicament is selected from the group consisting of antibiotics, pain relievers, pharmaceuticals, over-the-counter remedies or medicines, antacids, homeopathic remedies, herbal remedies, folk remedies, antimicrobials, anti-inflammatory, and anti-diarrheals.

3. A film as recited in claim 1 wherein said medicine or medicament is present in an edible quantity of said film in a pharmaceutically effective amount.

4. A film as recited in claim 1 wherein said medicine or medicament is present in said film in an amount that is not governmentally recognized as being pharmaceutically effective.

5. A film as recited in claim 1 wherein said film further comprises an ingredient selected from the group consisting of acid, tartness flavorings, sour flavorings, and sweeteners.

6. A film as recited in claim 1 wherein said film includes at least two distinct flavorings.

7. A film as recited in claim 1 wherein upon disintegration in a human mouth, said film does not leave substantial residue that can which the consumer feels needs to be ejected from the mouth.

8. A film as recited in claim 1 wherein said film includes a film base that carries said medicine or medicament for delivery to a human consumer.

9. A film as recited in claim 8 wherein said film base constitutes less than 50% of the mass of the film.

10. A film as recited in claim 8 wherein said film base constitutes more than 50% of the mass of the film.

11. A film as recited in claim 8 wherein said medicine or medicament constitutes at least 25% of the mass of the film

12. A film as recited in claim 1 wherein said film has a dosage of a single medicine or medicament.

13. A film as recited in claim 1 wherein said film has a dosage of a plurality of medicines or medicaments.

14. An film as recited in claim 1 wherein said film has a thickness of less than 200 microns.

15. A film as recited in claim 1 wherein said film has a thickness of les than 100 microns.

16. A vitamin-containing orally soluble edible film comprising:
one or more layers of film,
said film being orally soluble such that it disintegrates quickly upon placement in a human mouth without leaving substantial residue that can be felt by the human tongue or which needs to be swallowed or ejected from the mouth,
said film being edible so that it may pass through a human digestive system,
said film being constructed so that it may consumed without creating noise that is readily detected by a person other than the person consuming the snack,
said film being of small bulk so that it may be consumed without swallowing,
said film being of a construction that permits it to be consumed without creating debris, stickiness or mess outside of the human body,
at least one flavoring present in said film, said flavoring providing a flavor sensation to a person who places the snack on his or her tongue in order to satisfy a craving or provide flavor satisfaction to a consumer, and
at least one medicine or medicament carried by said film.

17. A film as recited in claim 16 wherein said medicine or medicament is selected from the group consisting of antibiotics, pain relievers, pharmaceuticals, medicines, antacids, antimicrobials, anti-inflammatories, anti-virals, anti-fungal and anti-diahrreals.

18. A film as recited in claim 17 wherein said medicine or medicament is present in an edible quantity of said film in a pharmaceutically effective amount.

19. A film as recited in claim 16 wherein said medicine or medicament is present in said film in an amount that is not governmentally recognized as being pharmaceutically effective.

20. A film as recited in claim 16 wherein said film further comprises an ingredient selected from the group consisting of acid, tartness flavorings, sour flavorings, and sweeteners.

21. A film as recited in claim 16 wherein said film includes at least two distinct flavorings.

22. A film as recited in claim 16 wherein upon disintegration in a human mouth, said film does not leave substantial residue that can which the consumer feels needs to be ejected from the mouth.

23. A film as recited in claim 16 wherein said film includes a film base that carries said medicine or medicament for delivery to a human consumer.

24. A film as recited in claim 23 wherein said film base constitutes less than 50% of the mass of the film.

25. A film as recited in claim 23 wherein said film base constitutes more than 50% of the mass of the film.

26. A film as recited in claim 23 wherein said medicine or medicament constitutes at least 25% of the mass of the film

27. A film as recited in claim 17 wherein said film has a dosage of a single medicine or medicament.

28. A film as recited in claim 17 herein said film has a dosage of a plurality of medicines or medicaments.

29. A film as recited in claim 17 wherein said film has a thickness of less than 200 microns.

30. A film as recited in claim 17 wherein said film has a thickness of les than 100 microns.

31. A film as recited in claim 17 further comprising:
at least one flavoring present in said film, said flavoring providing a flavor sensation to a person who places the snack on his or her tongue in order to satisfy a craving or provide flavor satisfaction to a consumer, said flavoring being the dominant flavoring in the snack, said dominant flavoring giving a stronger flavor sensation than other flavorings which may be present in the snack, and said dominant flavoring being a separate flavoring from any masking flavoring and enhancement flavoring that may be present in the snack, and said flavoring serving to mask any flavor which may be inherent in said medicine or medicament.
